(19) United States
(12) Patent Application Publication
(10) Pub. No.: US 2019/0069842 A1
(43) Pub. Date: Mar. 7, 2019

(54) WRIST BOUND ULTRASOUND-ON-A-CHIP DEVICE

(71) Applicant: Butterfly Network, Inc., Guilford, CT (US)

(72) Inventors: Jonathan M. Rothberg, Guilford, CT (US); Gregg Fergus, Stoughton, WI (US); Keith G. Fife, Palo Alto, CA (US); Tyler S. Ralston, Clinton, CT (US); Nevada J. Sanchez, Guilford, CT (US); Jaime Scott Zahorian, Guilford, CT (US); Kailiang Chen, Branford, CT (US); Christopher Thomas McNulty, Guilford, CT (US)

(21) Appl. No.: 16/122,956

(22) Filed: Sep. 6, 2018

Related U.S. Application Data
(60) Provisional application No. 62/555,494, filed on Sep. 7, 2017.

(51) Int. Cl. A61B 5/00; A61B 8/00; A61B 8/02; A61B 8/04; A61B 5/021

(52) U.S. Cl. CPC A61B 5/681; A61B 8/4494; A61B 5/021; A61B 8/02; A61B 8/04; A61B 8/4472

(57) ABSTRACT

Aspects of the technology described herein relate to an apparatus including an ultrasound-on-a-chip device configured to be bound to a user's wrist. The ultrasound-on-a-chip device may include a two-dimensional array of ultrasonic transducers. The transducers may be capacitive micromachined ultrasonic transducers (CMUTs) and may be configured to emit ultrasound waves having a frequency between approximately 5-20 MHz. A coupling strip may be coupled to the ultrasound-on-a-chip device to reduce the air gap between the ultrasound-on-a-chip device and the user's wrist. The ultrasound-on-a-chip device may be waterproof and may be able to perform both transverse and longitudinal ultrasound scanning without being rotated. The ultrasound-on-a-chip device may be configured to calculate pulse wave velocity through a blood vessel in a user's wrist.

### CROSS-REFERENCE TO RELATED APPLICATIONS

This application claims the benefit under 35 U.S.C. § 119(e) of U.S. Patent Application Ser. No. 62/555,494, filed Sep. 7, 2017 under Attorney Docket No. B1348.70056US00 and entitled “WRIST BOUND ULTRASOUND-ON-CHIP DEVICE,” which is hereby incorporated herein by reference in its entirety.

### FIELD

Generally, the aspects of the technology described herein relate to ultrasound systems. Some aspects relate to wrist bound ultrasound systems.

### BACKGROUND

Ultrasound devices may be used to perform diagnostic imaging and/or treatment, using sound waves with frequencies that are higher with respect to those audible to humans. Ultrasound imaging may be used to see internal soft tissue body structures, for example to find a source of disease or to exclude any pathology. When pulses of ultrasound are transmitted into tissue (e.g., by using a probe), sound waves are reflected off the tissue with different tissues reflecting varying degrees of sound. These reflected sound waves may then be recorded and displayed as an ultrasound image to the operator. The strength (amplitude) of the sound signal and the time it takes for the wave to travel through the body provide information used to produce the ultrasound image. Many different types of images can be formed using ultrasound devices, including real-time images. For example, images can be generated that show two-dimensional cross-sections of tissue, blood flow, motion of tissue over time, the location of blood, the presence of specific molecules, the stiffness of tissue, or the anatomy of a three-dimensional region.

### SUMMARY

According to one aspect, an apparatus includes an ultrasound-on-a-chip device configured to be bound to a user's wrist. In some embodiments, the ultrasound-on-a-chip device is waterproof. In some embodiments, the ultrasound-on-a-chip device is configured to perform both transverse and longitudinal ultrasound scanning of a blood vessel in the user's wrist without being rotated relative to the user's wrist. In some embodiments, the ultrasound-on-a-chip device includes a two-dimensional array of capacitive micromachined ultrasonic transducers (CMUTs). In some embodiments, the ultrasound-on-a-chip device includes a plurality of capacitive micromachined ultrasonic transducers (CMUTs) configured to emit ultrasound waves having a frequency between approximately 5-20 MHz.

In some embodiments, the apparatus further includes at least one wristband, an ultrasound module containing the ultrasound-on-a-chip device and coupled to the at least one wristband, and a coupling strip coupled to the ultrasound module and configured to couple the ultrasound module to the user's wrist. In some embodiments, the apparatus further includes a primary module coupled to the at least one wristband. In some embodiments, the primary module includes a display screen configured to display at least one of ultrasound data collected by the ultrasound-on-a-chip device, an ultrasound image generated from the ultrasound data, and data generated from the ultrasound data.

In some embodiments, the apparatus further includes at least one wristband configured to couple to a wristband of a wrist device; an ultrasound module containing the ultrasound-on-a-chip device and coupled to the at least one wristband; and a coupling strip coupled to the ultrasound module and configured to couple the ultrasound module to the user's wrist. In some embodiments, the apparatus further includes a connection cable extending externally from the at least one wristband and configured to electrically connect the ultrasound module to the primary module.

In some embodiments, the apparatus further includes at least one wristband, a primary module containing the ultrasound-on-a-chip device and coupled to the at least one wristband, and a coupling strip coupled to the primary module and configured to couple the primary module to the user's wrist.

In some embodiments, the apparatus further includes a reservoir containing liquid or gel and configured to refresh the coupling strip. In some embodiments, the reservoir includes a valve opening from the reservoir into the coupling strip and configured to enable flow of the liquid or gel from the reservoir to the coupling strip. In some embodiments, the valve is configured to enable flow of the liquid or gel from the reservoir to the coupling strip in response to mechanical pressure applied to a portion of the apparatus. In some embodiments, the apparatus further includes processing circuitry configured to automatically trigger the valve to enable flow of the liquid or gel from the reservoir to the coupling strip. In some embodiments, the reservoir further includes an input port configured to enable refilling of the reservoir with the liquid or gel.

In some embodiments, the apparatus further includes processing circuitry configured to generate a notification to reposition the ultrasound-on-a-chip device. In some embodiments, the apparatus further includes processing circuitry configured to generate a notification to replace the coupling strip or refresh the coupling strip with liquid or gel.

In some embodiments, the ultrasound-on-a-chip device is configured to transmit ultrasound data collected by the ultrasound-on-a-chip device to processing circuitry that is configured to analyze the ultrasound data using deep learning models. In some embodiments, the processing circuitry is configured to retrieve, from a server, ultrasound data collected by other ultrasound-on-a-chip devices and to use the ultrasound data collected by the other ultrasound-on-a-chip devices when training the deep learning models.

In some embodiments, the ultrasound-on-a-chip device is configured to transmit ultrasound data of a blood vessel to processing circuitry that is configured to calculate pulse wave velocity in the blood vessel based on the ultrasound data of the blood vessel. In some embodiments, the apparatus further includes a button and processing circuitry configured to trigger collection of ultrasound data by the ultrasound-on-a-chip device upon activation of the button.

According to another aspect, an apparatus includes a wristband and an ultrasound-on-a-chip device coupled to the wristband. In some embodiments, the wristband includes an interior surface and an exterior surface, and the ultrasound-on-a-chip device is positioned on the interior surface of the wristband.

According to another aspect, a method includes receiving ultrasound data collected from a user's wrist using an apparatus including at least one wristband, an ultrasound module containing the ultrasound-on-a-chip device and coupled to the at least one wristband, and a coupling strip coupled to the ultrasound module and configured to couple the ultrasound module to the user's wrist.

In some embodiments, the apparatus further includes a button, and the method further includes triggering collection of the ultrasound data based on activation of the button. In some embodiments, the method further includes determining whether a current amount of liquid or gel associated with the coupling strip is below a threshold amount, and based on determining that the current amount of liquid or gel associated with the coupling strip is below the threshold amount, generating a notification to replace the coupling strip or refresh the coupling strip with liquid or gel.

In some embodiments, the apparatus further includes a reservoir containing liquid or gel and a valve opening from the reservoir into the coupling strip and configured to enable flow of the liquid or gel from the reservoir to the coupling strip, and the method further includes determining whether a current amount of liquid or gel associated with the coupling strip is below a threshold amount, and based on determining that the current amount of liquid or gel associated with the coupling strip is below the threshold amount, triggering the valve to enable flow of the liquid or gel from the reservoir to the coupling strip. In some embodiments, determining whether the current amount of liquid or gel associated with the coupling strip is below the threshold amount includes performing an ultrasound scan. In some embodiments, determining whether the current amount of liquid or gel associated with the coupling strip is below the threshold amount includes using at least one of a moisture sensor, a capacitive sensor, and a skin conductivity sensor.

In some embodiments, the method further includes determining whether a current deviation of the ultrasound-on-a-chip device from a desired position exceeds a threshold deviation, and based on determining that the current deviation of the ultrasound-on-a-chip device from the desired position exceeds the threshold deviation, generating a notification to reposition the ultrasound-on-a-chip device.

In some embodiments, the apparatus further includes a display screen, and the method further includes generating for display, on the display screen, at least one of the ultrasound data, an ultrasound image generated from the ultrasound data, and data generated based on the ultrasound data.

According to another aspect, a method for calculating pulse wave velocity in a blood vessel includes: receiving, from an ultrasound-on-a-chip device configured to be bound to a user's wrist, first ultrasound data from a first ultrasound scan of the blood vessel in the user's wrist; calculating, based on the first ultrasound data, a cross-sectional area of the blood vessel; receiving, from the ultrasound-on-a-chip device, second ultrasound data from a second ultrasound scan of the blood vessel in the user's wrist, without rotating the ultrasound-on-a-chip device relative to the user's wrist between the first ultrasound scan and the second ultrasound scan; calculating, based on the second ultrasound data, volumetric blood flow through the blood vessel; and calculating, based on the cross-sectional area of the blood vessel and the volumetric blood flow, the pulse wave velocity in the blood vessel.

In some embodiments, the ultrasound-on-a-chip device is configured to use a two-dimensional array of ultrasound transducers to perform the first and second ultrasound scans. In some embodiments, the first ultrasound scan includes a transverse ultrasound scan of the blood vessel. In some embodiments, the second ultrasound scan includes a longitudinal ultrasound scan with an azimuthal steer towards the blood vessel. In some embodiments, the second ultrasound scan includes a transverse ultrasound scan with an elevational steer towards the blood vessel. In some embodiments, at least one of the first and second ultrasound scans includes using an ultrasound beam profile that is steered along a path that is not perpendicular or parallel to an azimuth or elevation direction of the two-dimensional array of ultrasound transducers. In some embodiments, the method further includes estimating a blood pressure in the blood vessel based on the pulse wave velocity in the blood vessel.

According to another aspect, a method for estimating blood pressure in a blood vessel includes measuring an elasticity of the blood vessel using an ultrasound-on-a-chip device configured to be bound to a user's wrist.

### BRIEF DESCRIPTION OF THE DRAWINGS

Various aspects and embodiments will be described with reference to the following exemplary and non-limiting figures. It should be appreciated that the figures are not necessarily drawn to scale. Items appearing in multiple figures are indicated by the same or a similar reference number in all the figures in which they appear.

FIG. 1 shows an example of an apparatus for an ultrasound-on-a-chip device configured to be worn on a user's wrist, in accordance with certain embodiments disclosed herein;

FIG. 2 shows an example of a user's dorsal wrist and the user's volar wrist when the user wears the assembled apparatus of FIG. 1;

FIG. 3 shows another example of an apparatus for an ultrasound-on-a-chip device configured to be worn on a user's wrist, in accordance with certain embodiments disclosed herein;

FIG. 4 shows another example of an apparatus for an ultrasound-on-a-chip device configured to be worn on a user's wrist, in accordance with certain embodiments disclosed herein;

FIG. 5 shows another example of an apparatus for an ultrasound-on-a-chip device configured to be worn on a user's wrist, in accordance with certain embodiments disclosed herein;

FIGS. 6A-6G show examples of an apparatus for an ultrasound-on-a-chip device configured to be bound to a user's wrist when the apparatus is assembled and worn;

FIG. 7 shows an example of an apparatus when electrically coupled to a user's personal wrist device;

FIG. 8 shows an example in which an ultrasound module includes reservoirs for refreshing a coupling strip in accordance with certain embodiments described herein;

FIG. 9 shows an example of recesses incorporated into an ultrasound module in accordance with certain embodiments disclosed herein;

FIG. 10 shows an example of a mechanical button incorporated into an ultrasound module in accordance with certain embodiments disclosed herein;

FIG. 11 shows an example of a virtual button on a display screen of a primary module in accordance with certain embodiments disclosed herein;

FIG. 12 shows an example of a mechanical button on a primary module in accordance with certain embodiments disclosed herein;

FIG. 13 shows an illustration of performing a transverse ultrasound scan of a blood vessel with a two-dimensional array of ultrasound transducers in accordance with certain embodiments disclosed herein;

FIG. 14 shows an illustration of performing a transverse ultrasound scan, with an elevational steer, of a blood vessel using the two-dimensional array of ultrasound transducers in accordance with certain embodiments disclosed herein;

FIG. 15 shows an illustration of performing a longitudinal ultrasound scan of a blood vessel with the two-dimensional array of ultrasound transducers in accordance with certain embodiments disclosed herein;

FIG. 16 shows an illustration of performing a longitudinal ultrasound scan, with an azimuthal steer, of a blood vessel using the two-dimensional array of ultrasound transducers in accordance with certain embodiments disclosed herein;

FIG. 17 shows an illustration of performing a transverse ultrasound scan of a blood vessel using the two-dimensional array of ultrasound transducers, when the blood vessel does not lie either perpendicular or parallel to the azimuth direction or the elevation direction, in accordance with certain embodiments disclosed herein;

FIG. 18 shows an example process for obtaining ultrasound data from a user's wrist, in accordance with certain embodiments disclosed herein; and

FIG. 19 shows an example process for calculating pulse wave velocity (PWV), in accordance with certain embodiments disclosed herein.

### DETAILED DESCRIPTION

Conventional ultrasound systems are large, complex, and expensive systems that are typically only purchased by large medical facilities with significant financial resources. Recently, cheaper and less complex ultrasound imaging devices have been introduced. Such imaging devices may include ultrasonic transducers monolithically integrated onto a single semiconductor die to form a monolithic ultrasound device. Aspects of such ultrasound-on-a chip devices are described in U.S. patent application Ser. No. 15/415,434 titled “UNIVERSAL ULTRASOUND DEVICE AND RELATED APPARATUS AND METHODS,” filed on Jan. 25, 2017 (and assigned to the assignee of the instant application) and published as U.S. Pat. Pub. No. 2017/0360397 A1, which is incorporated by reference herein in its entirety. The reduced cost and increased portability of these new ultrasound devices may make them significantly more accessible to the general public than conventional ultrasound devices.

Although the reduced cost and increased portability of ultrasound imaging devices make them more accessible to the general populace, people who could make use of such devices may have little to no training for how to use them. The inventors have recognized that a wrist bound ultrasound-on-a-chip device may be helpful in minimizing complexity of collecting ultrasound data. Instead of placing an ultrasound probe on a user every time collection of ultrasound data is needed, the ultrasound-on-a-chip device may be kept bound to the wrist for an extended period of time (e.g., 1 hour, 6 hours, 12 hours, 1 day, 1 week, 1 month, indefinitely, or any suitable length of time), in place for collection of ultrasound data when needed. Instead of a user actively initiating collection of ultrasound data when needed, because the ultrasound-on-a-chip device is kept bound to the wrist for an extended period of time (e.g., 1 hour, 6 hours, 12 hours, 1 day, 1 week, 1 month, indefinitely, or any suitable length of time) and in place for collection of ultrasound data when needed, the ultrasound-on-a-chip device may be able to automatically initiate collection of ultrasound data, without requiring active initiation of data collection by a user. Additionally, it may not be necessary for a medical professional knowledgeable about ultrasound data collection to be involved in collection of ultrasound data with the wrist bound ultrasound-on-a-chip device. The ultrasound data may be collected and sent to one or more servers (also known as a “cloud”), from which a user and/or his or her medical professional can retrieve the ultrasound data and track the progress of ultrasound scans. Furthermore, blood pressure or other metrics may be learned in a deep learning framework applied to aggregated data and an inference can be run on the ultrasound data that has been uploaded to the server(s). The wrist bound ultrasound-on-a-chip device may already be in place at the location on the wrist for ultrasound data collection, and parameters needed for ultrasound data collection may already be programmed into the ultrasound-on-a-chip device or automatically receivable by the ultrasound-on-a-chip from an external source. Because the ultrasound-on-a-chip device may be coupled to a wristwatch device or a bracelet, which the user may already wear, the wrist bound ultrasound-on-a-chip device may not require the user to wear an additional device in order to enable collection of ultrasound data from the wrist. A wrist bound ultrasound-on-a-chip device, with a form factor similar to a normal wristwatch or bracelet, may be comfortable and familiar for a user. When the ultrasound-on-a-chip device is part of an apparatus including a primary module such as a smartwatch, the primary module may provide functionality in combination with the ultrasound-on-a-chip device. For example, the user may view data collected by the ultrasound-on-a-chip device on a display screen of the primary module, the user may receive notifications about the ultrasound-on-a-chip device from the primary module (e.g., on a display screen of the primary module or by an audio speaker of the primary module), and the user may control operation of the ultrasound-on-a-chip device using physical and/or virtual buttons of the primary module.

The inventors have further recognized that the wrist may be an advantageous location for an ultrasound-on-a-chip device because useful ultrasound data may be collected from the wrist. For example, measurements of blood flow, heart rate, blood pressure, blood vessel diameter, and pulse wave velocity may be measured/calculated/estimated based on ultrasound data collected from the wrist.

The inventors have further recognized that an ultrasound-on-a-chip device that includes a two-dimensional array of ultrasound transducers may be helpful for applications involving collection of ultrasound data from the wrist. A two-dimensional array of ultrasound transducers can, for example, perform transverse and longitudinal ultrasound scanning and steer ultrasound beam profiles in the azimuthal and elevational directions, as well as steer ultrasound beam profiles in arbitrary orientations. This flexibility can be useful, for example, in applications requiring collection of multiple types of data that may require or be enabled by multiple ultrasound beam profiles and multiple scanning directions. For example, measuring PWV at the wrist may require collecting ultrasound data for measuring blood vessel diameter, spatial mean velocity, and/or blood vessel wall velocity, which may be enabled by the flexibility of a two-dimensional ultrasound transducer array.

The inventors have further recognized that using capacitive micromachined ultrasonic transducers (CMUTs), which may be integrated with CMOS (complementary metal-oxide-semiconductor) circuitry and referred to as CMOS ultrasonic transducers (CUTs), in a wrist bound ultrasound-on-a-chip device may be advantageous. The ultrasound transducers in the wrist bound ultrasound-on-a-chip device may be configured to emit ultrasound waves having frequencies between 5-20 MHz in order to collect ultrasound data from arteries in the wrist. These frequencies may represent the optimal frequencies in terms of attenuation and resolution based on the depth of the arteries in the wrist below the skin surface. CMUTs may be advantageous compared with piezoelectric micromachined ultrasonic transducers (PMUTs) for applications using high frequencies (e.g., frequencies between 5-20 MHz) for reasons related to manufacturability and sensitivity. In terms of manufacturability, high frequency applications require small elements with tight pitch (i.e., the distance between the centers of adjacent transducers) and narrow kerfs (i.e., the gap between adjacent transducers). Certain manufacturing processes for PMUTs (e.g., using dicing and filling) can make it difficult to produce small elements with tight pitch and narrow kerfs with consistent results because of the small scales involved. In contrast, the manufacturing processes for CMUTs may make it easier to produce small elements with tight pitch and narrow kerfs. CMUTs also have high sensitivity. As discussed further below, CMUTs may include a cavity formed in a substrate with a membrane overlying the cavity. CMUTs may be especially sensitive when their cavities are small and membranes are thick, which is advantageous for high frequency applications.

The inventors have further recognized that including a coupling strip in the wrist bound ultrasound-on-a-chip device may be helpful for reducing the air gap between the ultrasound-on-a-chip device (more particularly, an ultrasound module containing the ultrasound-on-a-chip device) and the user's wrist. In particular, the coupling strip may be configured to establish acceptable impedance matching coupling for ultrasound signal transmission and reception. To reduce the air gap between the ultrasound-on-a-chip device and the user's wrist, the coupling strip may be configured to be flexible such that the coupling strip conforms to the irregular surface of the user's wrist. Conventionally, ultrasound gel is applied to the user's skin prior to collection of ultrasound data from the user, in order to reduce the air gap between the ultrasound-on-a-chip device and the user's wrist and to establish acceptable impedance matching coupling for ultrasound signal transmission and reception. However, ultrasound gel tends to dry up and consequently may not be effective after a period of time. Accordingly, applications that may require collection of ultrasound data (continuously or periodically) over a period of time longer than the period of time during which ultrasound gel is effective may not be able to use conventional ultrasound gel. For example, a wrist bound ultrasound-on-a-chip device, which may be configured to be worn and used for data collection for an extended period of time (e.g., 1 hour, 6 hours, 12 hours, 1 day, 1 week, 1 month, indefinitely, or any suitable length of time), may benefit from an alternative to ultrasound gel that can function for longer than ultrasound gel. The coupling strip described herein may be helpful in providing benefits of ultrasound gel, such as reducing the air gap between the ultrasound-on-a-chip device and the user's wrist and ensuring proper impedance matching coupling for ultrasound signal transmission and reception, while also avoiding the limited period of time during which ultrasound gel is effective. For example, the coupling strip may not dry up as quickly as conventional ultrasound gel. As another example, the coupling strip may be more easily replaceable (for example, by peeling off an old coupling strip and adhering a new coupling strip to the ultrasound-on-a-chip device and/or the user's skin) than removing old ultrasound gel and applying new ultrasound gel. As another example, the coupling strip may be refreshable by addition of liquid to the coupling strip if the coupling strip dries out.

The inventors have further recognized that configuring a wrist bound ultrasound-on-a-chip device to be waterproof may be helpful. For example, if the wrist bound ultrasound-on-a-chip device is waterproof, prior to collection of ultrasound data, the user may dip the ultrasound-on-a-chip device in water, run the ultrasound-on-a-chip device over water, and/or take a shower with the ultrasound-on-a-chip device to create a water layer between the ultrasound-on-a-chip device and the user's skin, and thereby establish proper impedance matching coupling for ultrasound signal transmission and reception.

As used herein, a wrist bound object or an object configured to be bound to the wrist should be understood to mean that the object is configured to remain located at or near a subject's wrist without external application of force. For example, an ultrasound-on-a-chip device coupled to a wristwatch or a bracelet that is worn on a user's wrist may be considered “wrist bound.”

As used herein, an “ultrasound-on-a-chip device” should be understood to mean a device including micromachined ultrasound transducers integrated with a semiconductor die containing integrated circuitry.

It should be appreciated that the embodiments described herein may be implemented in any of numerous ways. Examples of specific implementations are provided below for illustrative purposes only. It should be appreciated that these embodiments and the features/capabilities provided may be used individually, all together, or in any combination of two or more, as aspects of the technology described herein are not limited in this respect.

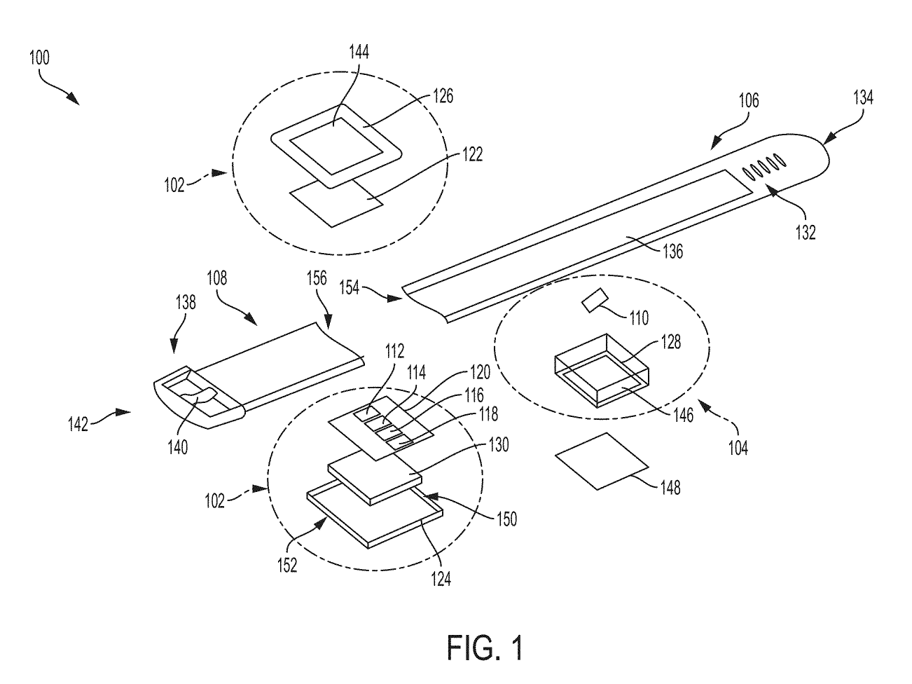

FIG. 1 shows an example of an apparatus 100 for an ultrasound-on-a-chip device configured to be worn on a user's wrist, in accordance with certain embodiments disclosed herein. In FIG. 1, the apparatus 100 is shown disassembled. The apparatus 100 is wearable by a user around the user's wrist and includes a primary module 102, an ultrasound module 104, a coupling strip 148, a first wristband 106, and a second wristband 108. It should be understood that as referred to herein, a “wristband” may be any type of band configured to encircle any portion of the wrist, or the entire wrist.

The ultrasound module 104 includes an ultrasound-on-a-chip device 110 and an ultrasound housing element 128. The primary module 102 includes a printed circuit board (PCB) 120, a display screen 122, a battery 130, and primary housing elements 124 and 126. On the PCB 120 is processing circuitry 112, memory circuitry 114, communication circuitry 116, and power management circuitry 118. The first wristband 106 includes a plurality of holes 132 at its first end portion 134 that are each located at a different distance from the first end portion 134 of the first wristband 106. Conductors 136 extend through the first wristband 106 and extend into the primary housing elements 124 and 126 to electrically connect the ultrasound module 104 to the PCB 120. The second wristband 108 includes a buckle 138 at its first end portion 142. The buckle 138 includes a pin 140.

The ultrasound-on-a-chip device 110 includes micromachined ultrasound transducers integrated with a semiconductor die containing integrated ultrasound circuitry. In some embodiments, the ultrasonic transducers may be formed on the same chip as the ultrasound circuitry to form a monolithic ultrasound device. In other embodiments, certain portions of the ultrasound circuitry may be in a different semiconductor chip than the transducers. The ultrasound transducers may be capacitive micromachined ultrasonic transducers (CMUTs). The CMUTs may be integrated with CMOS circuitry. A CMUT may, for example, include a cavity formed in a CMOS wafer, with a membrane overlying the cavity, and in some embodiments sealing the cavity. Electrodes may be provided to create a transducer cell from the covered cavity structure. The CMOS wafer may include integrated circuitry to which the transducer cell may be connected. The transducer cell and CMOS wafer may be monolithically integrated, thus forming an integrated ultrasonic transducer cell and integrated circuit on a single substrate (the CMOS wafer). CMUTs integrated with CMOS circuitry may be referred to as CMOS ultrasonic transducers (CUTs).

The ultrasound transducers may be arranged in a one-dimensional array or a two-dimensional array, and there may be 1024, 2048, 4096, 8192, 16384, or any other suitable number of transducer elements in the array. The transducers may be arranged with a 50 μm, 100 μm, 130 μm, 200 μm, 250 μm, or any other suitable pitch. The semiconductor die/dice may be 5 mm×5 mm, 10×5 mm, 1×1 cm, 1.5×1 cm, 1.5 cm×1.5 cm, 2×1 cm, 2×1.5 cm, 2×2 cm, or any other suitable size. In some embodiments, the ultrasound-on-a-chip device 110 includes a transducer array having 2048 transducer elements arranged in a 64×32 array with a 130 μm pitch on a semiconductor die that is 10×5 mm in size. In some embodiments, the ultrasound-on-a-chip device 110 includes a transducer array having 4096 transducer elements arranged in a 64×64 array with a 130 μm pitch on a semiconductor die that is 1×1 cm in size. The ultrasound circuitry in the ultrasound-on-a-chip device 110 may include transmit circuitry that transmits a signal to a transmit beamformer in the ultrasound-on-a-chip device 110 which in turn drives the ultrasound transducers to emit pulsed ultrasonic signals into the user's wrist. The pulsed ultrasonic signals may be back-scattered from structures in the user's wrist, such as blood vessels, to produce echoes that return to the transducers. These echoes may then be converted into electrical signals, or ultrasound data, by the transducer elements, and the electrical signals are received by receive circuitry in the ultrasound circuitry. The electrical signals representing the received echoes are sent to a receive beamformer in the ultrasound-on-a-chip device 110 that outputs ultrasound data in response to the received echoes. For further description of examples of ultrasound devices and ultrasound circuitry, see U.S. patent application Ser. No. 15/415,434 titled “UNIVERSAL ULTRASOUND DEVICE AND RELATED APPARATUS AND METHODS.”

In some embodiments, the ultrasound transducers in the ultrasound-on-a-chip device 110 may emit ultrasound waves having frequencies between approximately 5-20 MHz in order to collect ultrasound data from arteries in the wrist. These frequencies may represent the optimal frequencies in terms of attenuation and resolution based on the depth of the arteries in the wrist below the skin surface. In some embodiments, the ultrasound-on-a-chip device 110 may emit ultrasound waves having frequencies up to approximately 21 MHz, 22 MHz, 23 MHz, 24 MHz, 25 MHz, 26 MHz, 27 MHz, 28 MHz, 29 MHz, 30 MHz, >30 MHz, or any suitable frequency. In some embodiments, the ultrasound-on-a-chip device 110 may emit ultrasound waves having frequencies down to approximately 4 MHz, 3 MHz, 2 MHz, 1 MHz, <1 MHz, or any suitable frequency.

The ultrasound-on-a-chip device 110 is positioned in the ultrasound module 104 such that its longitudinal axis is parallel to the longitudinal axis of the first wristband 106. In the example of the radial artery, because the ultrasound-on-a-chip device 110 will be transverse to the radial artery when positioned on the wrist, it may be easier to position the ultrasound-on-a-chip device 110 over the radial artery, and not to the left or right of the radial artery, than if the ultrasound-on-a-chip device 110 is positioned with its longitudinal axis perpendicular to the first wristband 106. In some embodiments, it may be possible to rotate the ultrasound module 104 to a desired orientation relative to the first wristband 106 prior to coupling the ultrasound module 104 to the first wristband 106.

The ultrasound-on-a-chip device 110 may transmit collected ultrasound data over the conductors 136 to the processing circuitry 112. The ultrasound module 104 and the PCB 120 are electrically coupled to the conductors 136 which extend through the first wristband 106 and into the primary module 102. The conductors 136 may be, for example, in a flexible printed circuit board or a cable.

The ultrasound housing element 128 and the first wristband 106 enclose the ultrasound-on-a-chip device 110. The ultrasound housing element 128 has an acoustic lens 146 through which ultrasonic waves can propagate from the ultrasound-on-a-chip device 110 into the user's wrist. In some embodiments, the acoustic lens 146 is a simple opening in the ultrasound housing element 128. When the apparatus 100 is assembled, the ultrasound housing element 128 faces the user's wrist. In some embodiments, the ultrasound housing element 128 is a protrusion from the first wristband 106 that forms a cavity that contains the ultrasound-on-a-chip device 110.

The coupling strip 148 is attached to the surface of the acoustic lens 146 that faces the user's wrist. The coupling strip 148 is configured to reduce the air gap between the ultrasound module 104 and the user's wrist and to establish acceptable impedance matching coupling for ultrasound signal transmission and reception. In some embodiments, therefore, the coupling strip 148 may be considered an impedance matching strip, or an impedance matching coupler. Further examples of the coupling strip 148 are described in more detail hereinafter in the section entitled “Example Coupling Strips.”

In the primary module 102, the PCB 120 is communicatively coupled to the display screen 122, for example by internal wires within the primary housing elements 124 and 126, and includes processing circuitry 112, memory circuitry 114, communication circuitry 116, and power management circuitry 118, which may be included in one or more semiconductor chips on the PCB 120. The processing circuitry 112 may be configured to perform any of the functionality described herein. The processing circuitry 112 may include one or more processors (e.g., computer hardware processors) and may be configured to execute one or more processor-executable instructions stored in the memory circuitry 114. The memory circuitry 114 may be used for storing programs and data and may comprise one or more storage devices such as non-transitory computer-readable storage media. The processing circuitry 112 may control writing data to and reading data from the memory circuitry 114 in any suitable manner. The processing circuitry 112 is configured to receive ultrasound data from the ultrasound-on-a-chip device 110 and includes image reconstructions circuitry for reconstructing the ultrasound data into an ultrasound image (which may be two-dimensional images or, when the ultrasound-on-a-chip device 110 includes a two-dimensional array, three-dimensional images). The processing circuitry 112 may also be configured to perform calculations (e.g., anatomical or physiological measurements) based on ultrasound data and/or ultrasound images (which may be two-dimensional images or, when the ultrasound-on-a-chip device 110 includes a two-dimensional array, three-dimensional images). The processing circuitry 112 may include specially-programmed and/or special-purpose hardware such as an application-specific integrated circuit (ASIC). For example, the processing circuitry 112 may comprise one or more ASICs specifically designed for machine learning (e.g., deep learning). The ASICs specifically designed for machine learning may be employed to, for example, accelerate the inference phase of a neural network. The processing circuitry 112 also includes control circuitry that is configured to supply control signals that are transmitted over the conductors 136 to control operation of the ultrasound-on-a-chip device 110, such as operation of the transmit and receive circuitry. The control circuitry is also configured to supply control signals to the display screen 122, the circuitry on the PCB 120, and the ultrasound-on-a-chip device 110 to control their operation. The processing circuitry 112 may include a field-programmable gate array (FPGA).

The battery 130 is electrically connected to the PCB 120 and the display screen 122 to provide power to the circuitry on the PCB 120 and the display screen 122. The battery 130 is also configured to supply power to the ultrasound-on-a-chip device 110 over the conductors 136. The battery 130 may be any type of battery, such as a button cell battery (e.g., a zinc air cell battery, type PR48, size A13), a lithium ion battery, or a lithium polymer battery. The battery 130 may be rechargeable. The power management circuitry 118 is configured to manage supply of power from the battery 130 to the PCB 120, the display screen 122, and to the ultrasound-on-a-chip device 110. The power management circuitry 118 may be responsible for converting one or more input voltages from the battery 130 into voltages needed to carry out operation of the ultrasound-on-a-chip device 110, and for otherwise managing power consumption within the device ultrasound-on-a-chip device 110. For example, the power management circuitry 118 may step the input voltage up or down, as necessary, using a charge pump circuit or via some other DC-to-DC voltage conversion mechanism.

The communication circuitry 116 is configured to wirelessly transmit data (e.g., ultrasound data, ultrasound images, calculations based on ultrasound data/images) to an external device, such as external host device, workstation, or server. The communication circuitry 116 may include BLUETOOTH, ZIGBEE, and/or WiFi wireless communication circuitry. In some embodiments, the communication circuitry 116 may be configured to transmit data to the external device over a wired connection, such as a SERDES, DDR, USB, OR MIPI wired connection.

The primary module 102 may be configured as any type of electronic device and may perform functions unrelated to ultrasound data collection. For example, the primary module 102 may be configured as a smartwatch, and the display screen 122 may be configured to display any type of data, including the time, e-mail, instant messages, and/or the Internet. The display screen 122 may be any type of display screen, such as a low-power light emitting diode (LED) array, a liquid-crystal display (LCD) array, an active-matrix organic light-emitting diode (AMOLED) display, or a quantum dot display. The display screen 122 may be curved. The primary module 102 may include other sensors, such as global positioning, gyroscope, accelerometer, barometer, blood alcohol level, glucose level, blood oxygenation level, microphone, heart rate, ultraviolet, and galvanic skin response sensor, and the display screen 122 may display data from these additional sensors. In some embodiments, the display screen 122 may be absent.

In some embodiments, the ultrasound module 104 is configured to communicate with the primary module 102 wirelessly. In such embodiments, the ultrasound module 104 may include wireless communication circuitry configured to communicate wirelessly with the communication circuitry 116 of the primary module 102. The ultrasound module 104 and the primary module 102 may wirelessly communicate ultrasound data from the ultrasound module 104 to the primary module 102 and control signals from the primary module 102 to the ultrasound module 104. In some embodiments, the ultrasound module 104 includes a battery and does not draw power from the battery 130 in the primary module 102. In embodiments where the ultrasound module 104 communicates wirelessly with the primary module 102 and has its own battery, the conductors 136 may be absent. In some embodiments the ultrasound module 104 may charge or power itself inductively from the primary module 102 or an auxiliary charger.

In some embodiments, the ultrasound module 104 may include internal processing circuitry 112, memory circuitry 114, communication circuitry 116, and/or power management circuitry 118. Portions of the circuitry may be integrated with the ultrasound-on-a-chip device 110. In such embodiments, the ultrasound module 104 may perform image reconstruction and/or data transmission to an external device using circuitry internal to the ultrasound module 104, and may not communicate with the primary module 102. Accordingly, the conductors 136 may be absent.

The primary housing elements 124 and 126 enclose the PCB 120, the display screen 122, and the battery 130. The display screen 122 is positioned adjacent to the primary housing element 124, which includes an opening 144 through which the display screen 122 can be seen. When the apparatus 100 is assembled, the primary housing element 124 faces the user's wrist and the primary housing element 126 faces away from the user's wrist. The primary housing element 126 and the display screen 122 are positioned on an opposite surface of the apparatus 100 (i.e., the surface that faces away from the user's wrist) than the PCB 120, the battery 130, and the primary housing element 124. In some embodiments, the primary housing elements 124 and 126 may be a single element. For example, the single primary housing element may have a hinge so that the ultrasound housing element can open the PCB 120, the display screen 122, and the battery 130 can be inserted inside. As another example, the single primary housing element may have a slot into which the PCB 120, the display screen 122, and the battery 130 can be inserted.

The first wristband 106 is coupled at its second end portion 154 to a first end portion 150 of the primary housing element 124. The second wristband 108 is coupled at its second end portion 156 to a second end portion 152 of the primary housing element 124. The first and second wristbands 106 and 108 may be configured to couple to the primary housing element 124 through any coupling means, such as a clip, a snap, a screw, an adhesive, magnetism, hook and loop fastener (e.g., Velcro), an interlocking fit, etc. In some embodiments, the primary housing element 124 may include pairs of lugs at each of its first and second end portions 134 and 136, with spring bars bridging each pair of lugs, and the first and second wristbands 106 and 108 may loop around the spring bars. The first and second wristbands 106 and 108 may be made of any material, such as leather, fabric, plastic, and metal. The first and second wristbands 106 and 108 may have any shape and may resemble a conventional band for a wristwatch or a bracelet.

The apparatus 100 can be bound to the user's wrist by inserting the pin 140 into one of the plurality of holes 132. Based on which hole of the plurality of holes 132 is used, the circumference of the apparatus 100 can be adjusted so that the apparatus 100 fits around the user's wrist. In some embodiments, the apparatus 100 may be bound to the user's wrist using other mechanisms. For example, instead of the plurality of holes 132 and the buckle 138, the first and second wristbands 106 and 108 may include a clip, a snap, Velcro, magnets, or an interlocking fit. In some embodiments, the apparatus 100 includes just one wristband, or more than two wristbands.

The ultrasound module 104 is configured to attach to the first wristband 106. In some embodiments, the ultrasound module 104 is attached to the first wristband 106 at a position not intended to be moved. For example, the ultrasound module 104 may be positioned at a specific location on the first wristband 106 such that, when the apparatus 100 is worn, the ultrasound module 104 is positioned over a specific region of the user's wrist (e.g., the radial artery). The ultrasound module 104 may be configured to attach to the first wristband 106 through any coupling means. For example, the ultrasound module 104 may attach to the first wristband 106 through complementary Velcro, magnets, or snaps on the ultrasound module and the first wristband 106. In some embodiments, the apparatus is configured such that the position of the ultrasound module 104 on the first wristband 106 can be changed. In some embodiments, the first wristband 106 may include a plurality of discrete coupling points along its length (e.g., discrete magnets, discrete Velcro elements, discrete snap locations). In other embodiments, the first wristband 106 has a continuous coupling region along its length (e.g., a continuous length of magnetic material or a continuous length of hook and loop fastener (e.g., Velcro) material). In some embodiments, the ultrasound module 104 may include a clip for clipping the ultrasound module 104 to the first wristband 106. In other embodiments, the first wristband 106 may have a cavity into which the ultrasound-on-a-chip device 110 is placed. In yet other embodiments, the first wristband 106 includes a plurality of holes and the ultrasound module 104 includes a pin, and the ultrasound module 104 may be coupled to the first wristband 106 by inserting the pin into one of the plurality of holes.

In some embodiments, the primary module 102 may be absent, and the PCB 120, the processing circuitry 112, the memory circuitry 114, the communication circuitry 116, the power management circuitry 118, and the battery 130 may be included in the ultrasound module 104. In such embodiments, the first wristband 106 and the second wristband 108 may be a single continuous wristband.

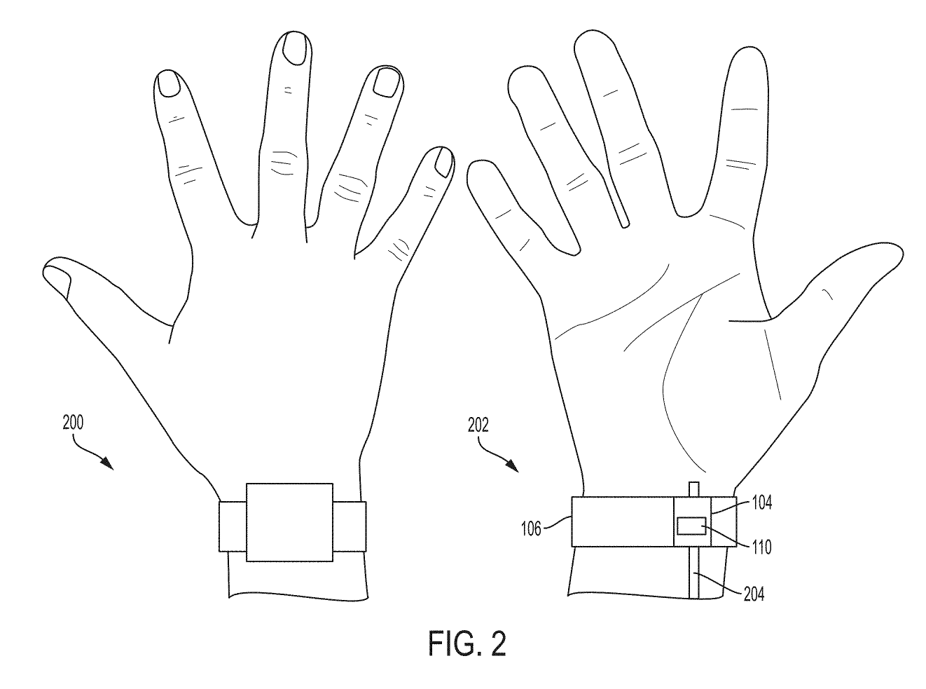

FIG. 2 shows an example of a user's dorsal wrist 200 and the user's volar wrist 202 when the user wears the assembled apparatus 100 of FIG. 1. The ultrasound module 104 is positioned on the first wristband 106 such that the ultrasound-on-a-chip device 110 (visible through the ultrasound module 104) is positioned above the radial artery 204. In particular, the ultrasound module 104 is positioned off-center towards the thumb on the portion of the first wristband 106 that contacts the user's volar wrist 202. At the radial artery, measurements of blood flow, heart rate, blood pressure, blood vessel diameter, and pulse wave velocity may be taken based on ultrasound data collected by the ultrasound-on-a-chip device 110 from the radial artery.

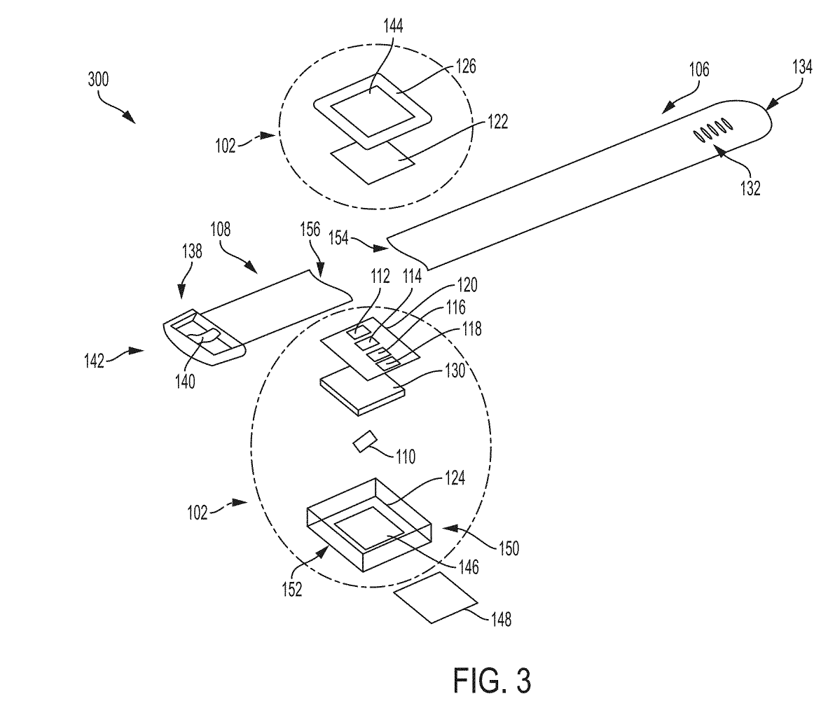

FIG. 3 shows another example of an apparatus 300 for an ultrasound-on-a-chip device configured to be worn on a user's wrist, in accordance with certain embodiments disclosed herein. The apparatus 300 is wearable by a user around the user's wrist. In FIG. 3, the apparatus 300 is shown disassembled. The following description discusses differences between the apparatus 300 and the apparatus 100.

The apparatus 300 lacks the ultrasound module 104. The ultrasound-on-a-chip device 110 is located in the primary module 102. The primary housing element 124 includes the acoustic lens 146, and the first wristband 106 lacks internal conductors to interface with an ultrasound module. The coupling strip 148 is coupled to the surface of the primary housing element 124 that faces the user's wrist. The ultrasound-on-a-chip device 110 and may be able to collect ultrasound data from various blood vessels (e.g., besides the radial artery, such as the anterior interosseous artery) depending on how the primary module 102 is worn (e.g., whether the primary module 102 is worn on the dorsal or volar wrist. Ultrasound data collected from veins may be used, for examine, to examine deep vein thrombosis, blockages to blood flow (such as clots), narrowing of vessels, tumors and congenital vascular malformations, reduced or absent blood flow, and greater than normal blood flow.

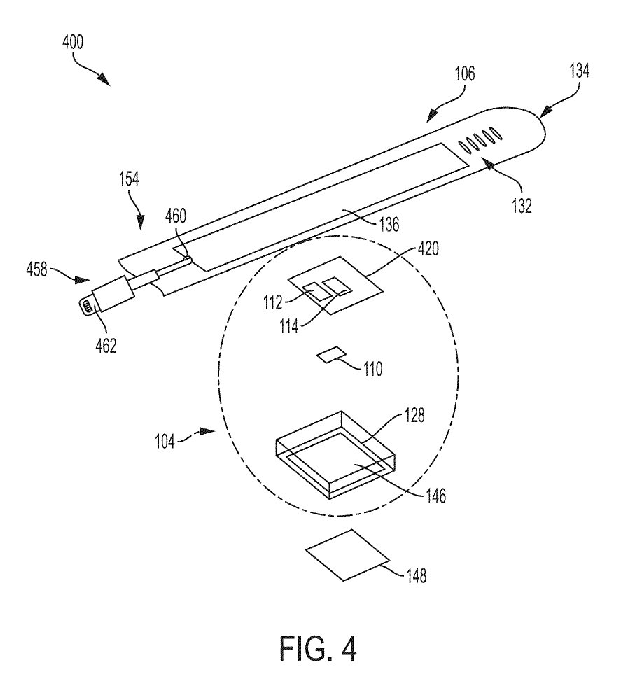

FIG. 4 shows another example of an apparatus 400 for an ultrasound-on-a-chip device configured to be worn on a user's wrist, in accordance with certain embodiments disclosed herein. The apparatus 400 is configured as a wristband that the user can physically couple to a wristband of his or her personal smartwatch module and electrically connect to the smartwatch module. In some embodiments, the apparatus 400 may be configured as an interchangeable wristband that the user can couple (physically and electrically) to his or her personal smartwatch module directly, replacing the smartwatch's original wristband. In FIG. 4, the apparatus 400 is shown disassembled. The following description discusses differences between the apparatus 400 and the apparatus 100.

The apparatus 400 lacks the primary module 102. The ultrasound module 104 includes a printed circuit board (PCB) 420. On the PCB 420 is the processing circuitry 112 and the memory circuitry 114. In contrast to apparatus 300, the ultrasound module 304 has internal processing circuitry 312 and memory circuitry 315, because the smartwatch to which the apparatus 300 is intended to be coupled may not have processing and memory circuitry capable of interfacing with the ultrasound-on-a-chip device 110 and processing ultrasound data.

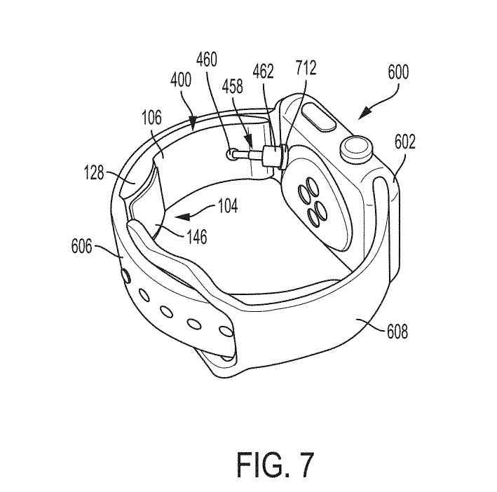

The first wristband 106 includes conductors 136 extending through the first wristband 106 that connect to a connection cable 458 at the second end portion 154 of the first wristband 106. The connection cable 458 exits from the first wristband 106 through an opening 460 in the first wristband 106 and has a male connector 462 configured to connect to a complementary female port on the user's personal smartwatch. Examples of plugging the male connector 462 into the smartwatch will be illustrated further in FIG. 7. In some embodiments, the apparatus 400 may include a plate configured to screw into the user's personal smartwatch at the complementary female port and to prevent the male connector 462 from being removed from the female port on the smartwatch during use of the apparatus 400.

The conductors 136 and the connection cable 458 electrically connect the ultrasound module 104 to the user's smartwatch. Accordingly, the ultrasound module 104 may use components within the user's smartwatch, and the ultrasound module 104 does not itself need to include these components. For example, in FIG. 400, the ultrasound module 104 is configured to draw power from the smartwatch's battery to power the ultrasound-on-a-chip device 110 and circuitry on the PCB 420. Additionally, the ultrasound module 104 is configured to transmit through the conductors 136 and the connection cable 458 data (e.g., ultrasound data, ultrasound images, calculations based on ultrasound images) to communication circuitry within the smartwatch for wireless transmission to an external device, such as external host device, workstation, or server. The user's personal smartwatch may run an application (“app”) configured to interface with the ultrasound module 104. The connection cable 458 may be any type of connection cable, such as a lightning connector or a mini-USB connector.

The apparatus 400 may be configured to couple along its longitudinal axis to the longitudinal axis of the smartwatch's wristband. The apparatus 400 may couple to the wristband of the user's personal smartwatch using any coupling means. For example, the first wristband 106 may include pins configured to be inserted into holes in the wristband of the user's smartwatch. As other examples, the apparatus 400 may couple to the wristband of the user's smartwatch with screws, hook and loop fastening (e.g., Velcro), adhesive, a snap, a slot and groove, one or more magnets. In embodiments where the apparatus 400 couples directly to the smartwatch module, replacing the smartwatch's wristband, the apparatus 400 may be configured to couple to the smartwatch module through any coupling means, such as a clip, a snap, a screw, an adhesive, magnetism, hook and loop fastening (e.g., Velcro), an interlocking fit, etc. In some embodiments, the smartwatch module may include pairs of lugs at each of its ends, with spring bars bridging each pair of lugs, and the first and second wristbands 106 and 108 may loop around the spring bars.

In some embodiments, the ultrasound module 104 has an internal battery and is not configured to draw on the battery in the user's smartwatch. In some embodiments, the ultrasound module 104 has communication circuitry internal to the ultrasound module 104 and is not configured to use communication circuitry in the user's smartwatch. In some embodiments, the ultrasound module 104 may transmit ultrasound data collected by the ultrasound-on-a-chip device 110 to processing circuitry in the user's smartwatch that is configured to reconstruct the ultrasound data into ultrasound images (which may be two-dimensional images or, when the ultrasound-on-a-chip device 110 includes a two-dimensional array, three-dimensional images), and may receive control signals from control circuitry in the user's smartwatch. For example, an application on the user's smartwatch may include instructions for the processing circuitry to reconstruct ultrasound data into ultrasound images and instructions for the control circuitry to output control signals for the ultrasound-on-a-chip device 110. In such embodiments, the ultrasound module 104 may lack the processing circuitry 112 and/or the memory circuitry 114.

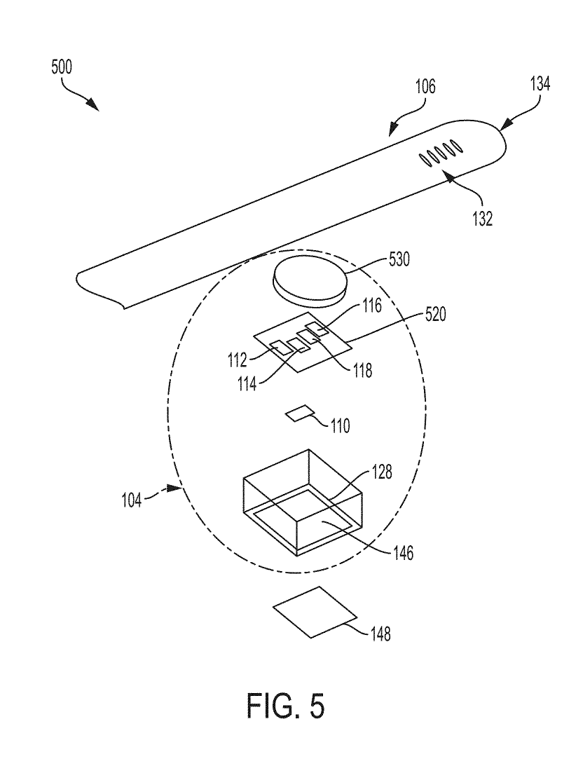

FIG. 5 shows another example of an apparatus 500 for an ultrasound-on-a-chip device configured to be worn on a user's wrist, in accordance with certain embodiments disclosed herein. The apparatus 500 is configured as a wristband that the user can physically couple to a wristband of his or her personal wrist device, which may be a standard analog watch module, a standard digital watch module, or a smartwatch. In some embodiments, the apparatus 500 may be configured as an interchangeable wristband that the user can couple (physically and electrically) to his or her personal wrist device, replacing the wrist device's original wristband. In FIG. 5, the apparatus 500 is shown disassembled. The following description discusses differences between the apparatus 500 and the apparatus 400.

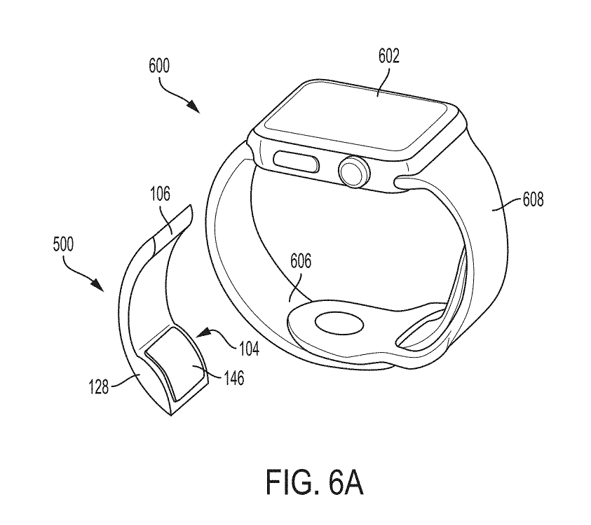

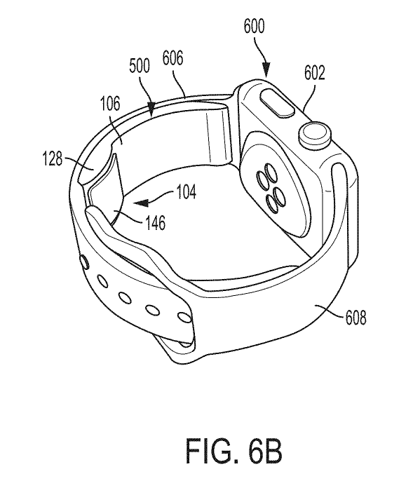

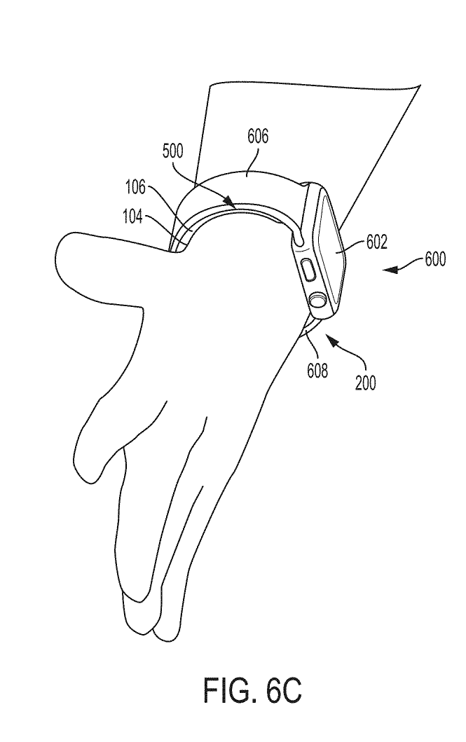

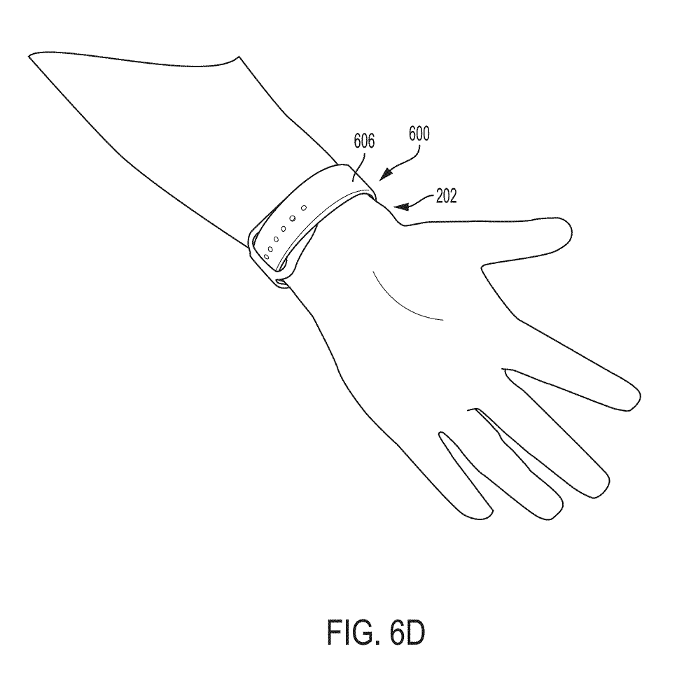

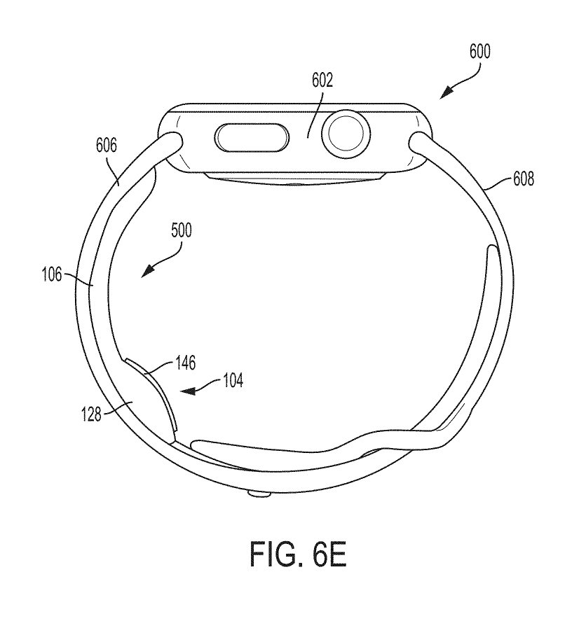

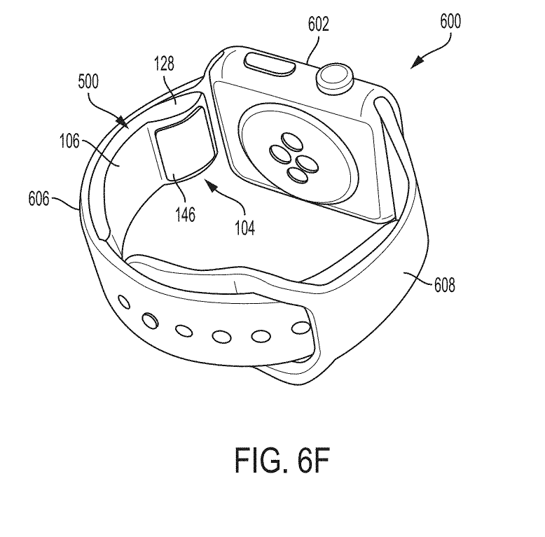

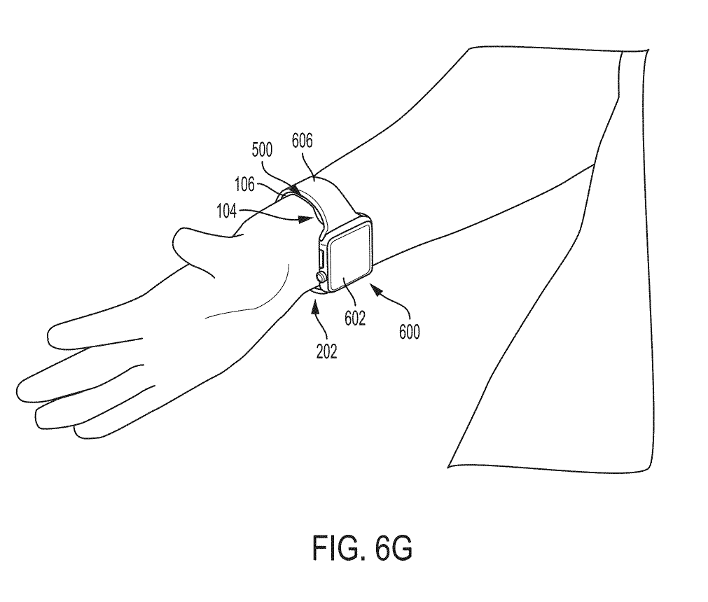

The ultrasound module 104 includes a printed circuit board (PCB) 520 and a battery 530. On the PCB 520 is the processing circuitry 112, the memory circuitry 114, the communication circuitry 116, and the power management circuitry 118. Accordingly, in contrast to apparatus 400, the ultrasound module 104 does not need to use components (e.g., communication circuitry, battery) outside of the ultrasound module 104 (e.g., in the user's personal smartwatch to which the apparatus 500 is coupled) because these components are already include internally in the ultrasound module 104. Therefore, the first wristband 106 lacks communication means (e.g., conductors internal to the first wristband 106 and a connection cable extending from the first wristband 106) to interface with the user's personal wristwatch. The battery 530 may be any type of battery, such as a button cell battery (e.g., a zinc air cell battery, type PR48, size A13), a lithium ion battery, or a lithium polymer battery. The battery 530 may be rechargeable. Examples of coupling the apparatus 500 to the wristband of the user's wrist device will be illustrated further in FIGS. 6A-6G.

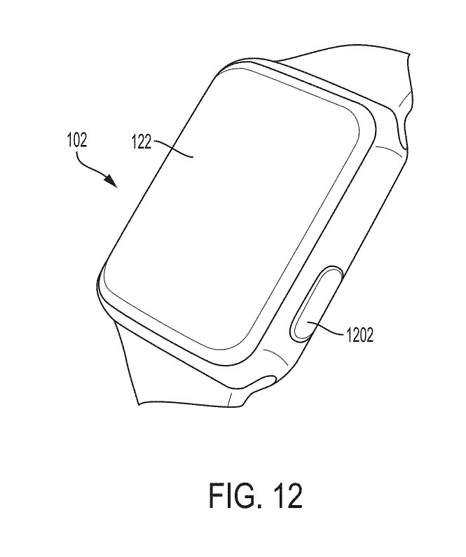

FIGS. 6A-6G show examples of an apparatus for an ultrasound-on-a-chip device configured to be bound to a user's wrist when the apparatus is assembled and worn. FIG. 6A shows the assembled apparatus 500 and a user's personal wrist device 600 prior to coupling the apparatus 500 to the wrist device 600. The apparatus 500 includes the ultrasound module 104, the first wristband 106, the ultrasound housing module 128, and the acoustic lens 146. The coupling strip 148 is not shown in FIG. 6A. The wrist device 600 includes a primary module 602, a first wristband 606, and a second wristband 608. The primary module includes a button that will be discussed further in relation to FIG. 12. FIG. 6B shows the apparatus 500 coupled to the wrist device 600. In particular, the first wristband 106 is coupled along its longitudinal axis to the first wristband 606 along its longitudinal axis. The apparatus 500 is oriented such that the ultrasound module 104 is distal from the primary module 602 of the wrist device 600. In this orientation, the ultrasound module 104 may be positioned over the radial artery when the wrist device 600 is worn with the primary module 602 on the dorsal wrist. FIGS. 6C and 6D show the apparatus 500 coupled to the wrist device 600 while being worn. FIG. 6C shows the dorsal wrist 200 and FIG. 6D shows the volar wrist 202. The apparatus 500 is oriented in the orientation of FIG. 6B, namely with the apparatus 500 oriented such that the ultrasound module 104 is distal from the primary module 602 of the wrist device 600. The wrist device 600 is worn with the primary module 602 on the dorsal wrist so that the ultrasound module 104 may be positioned over the radial artery. FIG. 6E shows a side view of the apparatus 500 coupled to the wrist device 600 in the orientation of FIG. 6B, namely with the apparatus 500 oriented such that the ultrasound module 104 is distal from the primary module 602 of the wrist device 600. FIG. 6F shows the apparatus 500 coupled to the wrist device 600 in a different orientation than in FIG. 6B. In particular, the apparatus 500 is coupled to the first wristband 606 such that the ultrasound module 104 is proximal to the primary module 602 of the wrist device 600. In this orientation, the ultrasound module 104 may be positioned over the radial artery when the wrist device 600 is worn with the primary module 602 on the volar wrist. FIG. 6G shows the apparatus 500 coupled to the wrist device 600 while being worn. The apparatus 500 is oriented in the orientation of FIG. 6F, namely with the apparatus 500 oriented such that the ultrasound module 104 is proximal to the primary module 602 of the wrist device 600. The wrist device 600 is worn with the primary module 602 on the volar wrist 202 so that the ultrasound module 104 may be positioned over the radial artery.

FIG. 7 shows an example of the apparatus 400 when electrically coupled to the user's personal wrist device 600. The wrist device 600 further includes a female port 712. The apparatus 400 includes the connection cable 458 that exits from the first wristband 106 through the opening 460 in the first wristband 106. The connection cable 458 has the male connector 462, which is plugged into the complementary female port 712 in the wrist device 600. In some embodiments, the connection cable 458 includes a female connector instead of, or in addition to, the male connector 462, and the wrist device 600 includes a male port (instead of or in addition to the female port 712) into which the female connector plugs. In some embodiments, a clasp on the wristband of the wrist device 600 has pins to which the connector (male or female) on the connection cable 458 may electrically couple.

FIGS. 1-7 show the ultrasound module 104 positioned on the apparatuses shown therein to face the user's wrist when the apparatuses are worn. In some embodiments, the ultrasound module is positioned on the apparatus to face away from the user's wrist when the apparatus is worn. For example, the ultrasound module 104 may be positioned on the outer surface of a wristband. Accordingly, a user may be able to move his or her wrist to place the ultrasound module 104 such that the ultrasound module 104 faces a portion of his or her body (e.g., heart, abdomen, uterus, etc.) in order to collect ultrasound data from that portion of the body. In such embodiments, the display screen 122 on the apparatus may display an ultrasound image/data generated based on collected ultrasound data while the user is holding the ultrasound module 104 at the desired location. For example, in the case of positioning the ultrasound module 104 over the heart, the display screen 122 may show an ultrasound image of the heart and/or display medical parameters such as ejection fraction, fractional shortening, ventricle diameter, ventricle volume, end-diastolic volume, end-systolic volume, cardiac output, stroke volume, intraventricular septum thickness, ventricle wall thickness, and pulse rate. The ultrasound image displayed on the display screen 122 may be a two-dimensional image or, when the ultrasound-on-a-chip device 110 includes a two-dimensional array, a representation of a three-dimensional image. In some embodiments, the display screen 122 may display instructions for guiding the user to position the ultrasound module 104 at the desired location while the user is moving his or her wrist. For further description of guiding a user in moving an ultrasound-on-a-chip device to a required position, see U.S. patent application Ser. No. 15/626,423 titled “AUTOMATIC IMAGE ACQUISITION FOR ASSISTING A USER TO OPERATE AN ULTRASOUND DEVICE,” filed on Jun. 19, 2017 (and assigned to the assignee of the instant application) and published as U.S. Pat. Pub. No. 2017-0360401 A1, which is incorporated by reference herein in its entirety.

In some embodiments, instead of or in addition to using a wristband to bind the ultrasound-on-a-chip device to the user's wrist, other means such as adhesives or clamps may be used.

Further description of data collection and processing with any of the apparatuses described herein are presented hereinafter in the section entitled “Example Data Collection and Processing.” Further description of system operations involving any of the apparatuses described herein are presented hereinafter in the section entitled “Example System Functions.” Further description of processes performed with any of the apparatuses described herein are presented hereinafter in the section entitled “Example Processes.” Further description of additional features that may be included in any of the apparatuses described herein are presented hereinafter in the section entitled “Example Apparatus Features.”

### Example Coupling Strips

As discussed above, the coupling strip 148 is configured to reduce the air gap between the ultrasound module 104 and the user's wrist. In particular, the coupling strip 148 is configured to couple to the acoustic lens 146 and establish acceptable impedance matching coupling for ultrasound signal transmission and reception. In some embodiments, therefore, the coupling strip 148 may be considered an impedance matching strip, or an impedance matching coupler. To reduce the air gap between the ultrasound module 104 and the user's wrist, the coupling strip 148 may be configured to be flexible such that the coupling strip 148 conforms to the irregular surface of the user's wrist.

In some embodiments, the coupling strip 148 includes a solid material and liquid absorbed within the solid material to increase the flexibility of the coupling strip 148. In some embodiments, the liquid includes a hydrophilic solution. In such embodiments, the coupling strip 148 may be configured to be refreshed with addition of water to the coupling strip 148 to reduce drying of the coupling strip and to maintain acceptable conformity of the coupling strip to the user's wrist. For example, the coupling strip 148 may be refreshed with water in a shower, by dipping the coupling strip 148 in water, or by running water over the coupling strip 148. In some embodiments, the coupling strip 148 includes a porous sponge that stores water and releases the water slowly, and can be refreshed with addition of water to the porous sponge. In some embodiments, the liquid includes a hydrophobic solution. In such embodiments, the coupling strip 148 is configured to be refreshed with oil, gel, or another hydrophobic consumable to reduce drying of the coupling strip 148 and maintain acceptable conformity of the coupling strip 148 to the user's wrist. In some embodiments, the apparatus (in particular, the ultrasound module 104 and the primary module 102) is configured to be waterproof so that if, for example, the ultrasound module 104 and the primary module 102 become wet while the coupling strip 148 is being refreshed, the ultrasound module 104 and the primary module 102 continue to function. For example, the ultrasound housing element 128 and the primary housing elements 124 and 126 may be waterproof housings.

In some embodiments, the coupling strip 148 is configured to be replaceable. For example, the coupling strip 148 may include an adhesive layer between the coupling strip 148 and the surface of the ultrasound module 104, and to replace the coupling strip 148, a user may peel the coupling strip 148 from the ultrasound module 104 and attach another coupling strip 148 to the ultrasound module 104.

Materials used in the coupling strip 148 may include a rubber material (which may be water-absorbent), a rubberized coating material, a silicone-based material, a gel-based material, an agar-based material, and a room-temperature-vulcanization silicone material. In some embodiments, the coupling strip 148 includes a rubbery silicone material that is sufficiently flexible to maintain acceptable contact with the user's wrist without requiring replacement. In some embodiments, the coupling strip 148 may include a spongy material that is capable of absorbing liquid and being refreshed with water (e.g., by splashing the coupling strip 148 with water, by dipping the coupling strip 148 in water, by taking a shower or bath, and/or by cleaning the coupling strip 148 with water) in order to maintain conformity of the coupling strip 148 to the user's wrist. In such embodiments, the spongy material may release the absorbed liquid at an acceptably low rate such that the coupling strip 148 requires refreshing at an acceptably low frequency.

In some embodiments, the ultrasound module 104 lacks a coupling strip, and the user may wet the wrist area (e.g., by dipping the wrist in water or running water over the first) prior to data collection to establish proper impedance matching coupling for ultrasound signal transmission and reception. Accordingly, the ultrasound module 104 can operate ultrasound gel-less. In such embodiments, the ultrasound module is configured to be waterproof. For example, the ultrasound housing element 128 may be a waterproof housing.

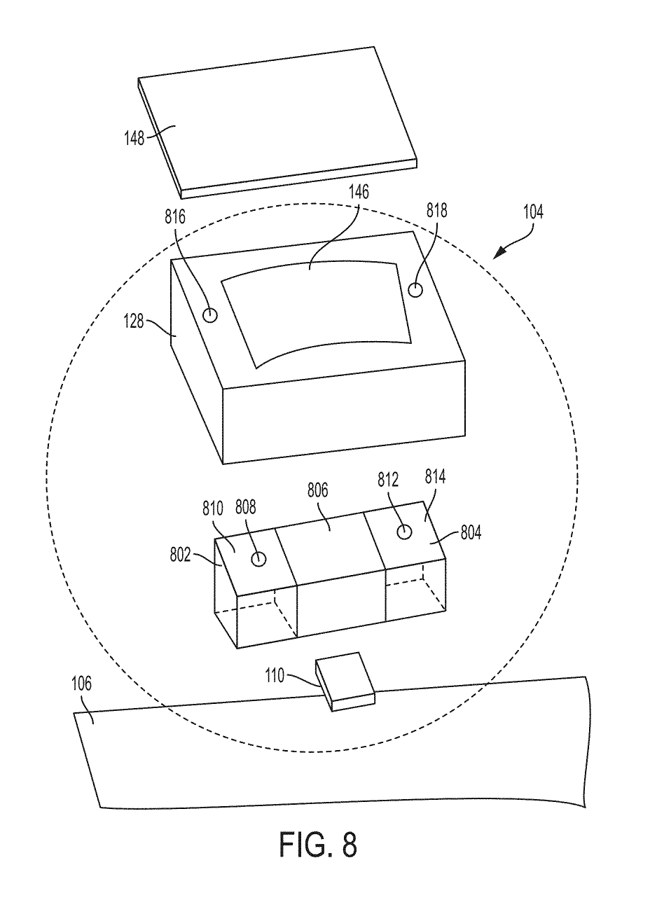

FIG. 8 shows an example in which the ultrasound module 104 includes reservoirs for refreshing the coupling strip 148 in accordance with certain embodiments described herein. In FIG. 8, the ultrasound module 104 includes the ultrasound-on-a-chip device 110, reservoirs 802 and 804, and cover 806. The reservoir 802 includes a valve 808 and a door 810. The reservoir 804 includes a valve 812 and a door 814. The ultrasound housing element 128 includes openings 816 and 818.

The ultrasound housing element 128 and the first wristband 106 enclose the reservoirs 802 and 804, the ultrasound-on-a-chip device 110, and the cover 806. The cover 806, which is hollow, covers the ultrasound-on-a-chip device 110 and, together with the ultrasound housing element 128, form an enclosure for the ultrasound-on-a-chip device 110. The coupling strip 148 is attached to the surface of the ultrasound housing element 128.

The valve 808 opens into the opening 816 and the valve 812 opens into the opening 818. The reservoirs 802 and 804 contain liquid or gel. The valve 808 is configured to release liquid or gel from the reservoir 802, through the opening 816, and into the coupling strip 148. The valve 812 is configured to release liquid or gel from the reservoir 802, through the opening 818, and into the coupling strip 148.

The liquid or gel in the reservoirs 802 and 804 may be hydrophilic or hydrophobic. As discussed above, the reservoirs 802 and 804 are configured to refresh the coupling strip 148 with the liquid or gel. In particular, the reservoirs 802 and 804 are configured to add the liquid or gel to the coupling strip 148, which may absorb the liquid or gel. Adding the liquid or gel to the coupling strip 148 may help to reduce drying of the coupling strip 148 and maintain acceptable conformity of the coupling strip 148 to the user's wrist.

The valves 808 and 812 may be mechanically or electrically activated. In some embodiments, the user may trigger the valves 808 and 812 to release liquid or gel from the reservoirs 802 and 804 into the coupling strip 148. In some embodiments, the user may apply mechanical pressure to the ultrasound module 104, either by directly applying mechanical pressure to the ultrasound module 104 or by applying mechanical pressure to another element to which the ultrasound module 104 is coupled (e.g., the first wristband 106), and the mechanical pressure may trigger the valves 802 and 812 to release at least a portion of the liquid or gel from the reservoirs 802 and 804 into the coupling strip 148. For example, mechanical pressure applied to the ultrasound module 104 may compress the reservoirs 802 and 804 and cause them to expel liquid or gel through the valves 808 and 812. In some embodiments, the user may apply the mechanical pressure to recesses in the first wristband 106. In some embodiments, the user may place his or her fingers over sensors on the first wristband 106 and the sensors may transmit an electrical signal to the valves 808 and 812 to release the liquid or gel from the reservoirs 802 and 804 into the coupling strip 148. In some embodiments, the user may activate a button (e.g., a mechanical button or a virtual button) and activation of the button may transmit an electrical signal to the valves 808 and 812 to release liquid or gel from the reservoirs 802 and 804 into the coupling strip 148.

In some embodiments, processing circuitry may be configured to automatically trigger the valves 808 and 812 to release the liquid or gel from the reservoirs 802 and 804 into the coupling strip 148. The processing circuitry may be processing circuitry 112 or processing circuitry in an external host device (e.g., a smartphone, tablet, or laptop), workstation, or server. For example, the processing circuitry may be configured to trigger the valves 808 and 812 to release the liquid or gel from the reservoirs 802 and 804 into the coupling strip 148 periodically. In some embodiments, the processing circuitry may be configured to trigger the valves 808 and 812 to release the liquid or gel from the reservoirs 802 and 804 into the coupling strip 148 based on detecting that the coupling strip 148 needs to be refreshed with liquid or gel. In some embodiments, detecting that the coupling strip 148 needs to be refreshed with liquid or gel includes determining whether a current amount of liquid or gel associated with the coupling strip 148 is below a threshold amount. In some embodiments, to detect that the coupling strip 148 needs to be refreshed with liquid or gel 424, the processing circuitry may be configured to analyze (continuously or periodically) ultrasound data collected by the ultrasound-on-a-chip device 110 and determine whether the collected ultrasound data shows signs (e.g., decreased quality of images) that the coupling strip 148 is conforming poorly to the user's wrist. In some embodiments, to detect that the coupling strip 148 needs to be refreshed with liquid or gel, the processing circuitry may be configured to receive signals from a moisture sensor in or adjacent to the coupling strip 148 indicating that the moisture level in or adjacent to the coupling strip 148 is below a threshold moisture level. In some embodiments, the processing circuitry may be configured to use other sensors to detect that the coupling strip 148 needs to be refreshed, such as capacitive sensors or skin conductivity sensors. In some embodiments, the processing circuitry may detect that the coupling strip 148 needs to be refreshed with liquid or gel and generate a notification that the user needs to refresh the coupling strip 148 with liquid or gel. In some embodiments, the notification may be displayed on the display screen 122. In some embodiments, the notification displayed on the display screen 122 may include text, an image, and/or a video. In some embodiments, the notification may include audio output from the primary module 102.

The door 810 can be opened to reveal an inside cavity of the reservoir 802 and enable refilling of the reservoir 802 with liquid or gel. The door 814 can be opened to reveal an inside cavity of the reservoir 804 and enable refilling of the reservoir 804 with liquid or gel. To refill the reservoirs 802 and 804, a user may remove the ultrasound housing element 128 from the first wristband 106, thereby revealing the reservoirs 802 and 804. The user may open the doors 810 and 814 and then run liquid or gel over the reservoirs 802 and 804, dip the reservoirs 802 and 804 into liquid or gel, or take a shower in order to add liquid to the reservoirs 802 and 804. The ultrasound-on-a-chip device 110 may be protected from damage during the refilling process by the cover 806, which forms an enclosure for the ultrasound-on-a-chip device 110, and may be waterproof. In some embodiments, the reservoirs 802 and 804 may be removable in order to allow the user to refill the reservoirs 802 and 804 without risking damage to the ultrasound-on-a-chip device 110. In some embodiments, the door 810 may be any type of input port.

In some embodiments, the reservoirs 802 and 804 may be coupled together as a single part, and/or may be connected together such that the reservoirs 802 and 804 constitute one reservoir. In some embodiments, one of the reservoirs 802 and 804 is absent, or there may be more than two reservoirs. In some embodiments, tubes may connect the reservoirs 802 and 804 to the coupling strip 148. In such embodiments, the reservoirs 802 and 804 may not be located adjacent to the coupling strip 148. In embodiments in which the ultrasound-on-a-chip device 110 is located within the primary module 102, the reservoirs 802 and 804 may be located within the primary module 102 as well. In some embodiments, the cover 806 may be absent. In some embodiments, other means for refilling the reservoirs 802 and 804 may be included, such as valves.

Other embodiments of reservoirs for refreshing the coupling strip 148 with liquid or gel are possible, such as reservoirs without valves. For example, in some embodiments, the reservoir includes an amorphous surface from which gel can be squeezed out like a sponge. In some embodiments, the reservoir includes a sponge-like material coupled through a restriction to the coupling strip 148 such that the reservoir may slowly release liquid or gel to refresh the coupling strip 148.

### Example Apparatus Features

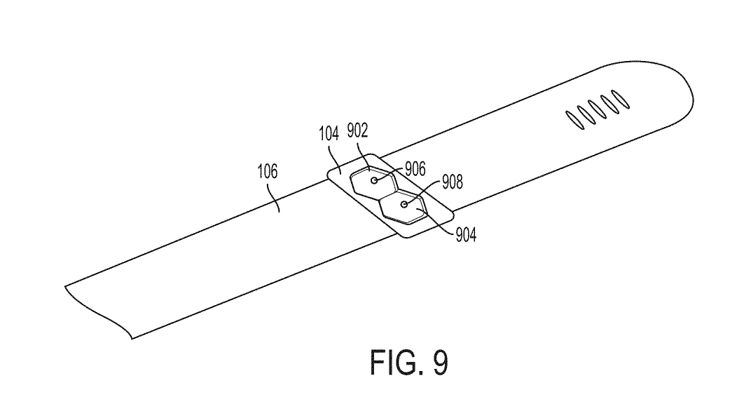

FIG. 9 shows an example of recesses incorporated into an ultrasound module in accordance with certain embodiments disclosed herein. FIG. 9 shows the ultrasound module 104 and the first wristband 106. The ultrasound module 104 is coupled to the first wristband 106 and incorporates inward recesses 902 and 904 on the outer surface of the ultrasound module 104 (i.e., the surface that faces away from the user's wrist). A sensor 906 is located within recess 902 and a sensor 908 is located within recess 904. The coupling strip 148 (not visible in FIG. 9) is coupled to the opposite surface of the ultrasound module 104 as the recesses 902 and 904. In some embodiments, a user may apply mechanical pressure with his or her fingers to the recesses 902 and 904. In such embodiments, the sensors 906 and 908 may be configured to detect the application of the mechanical pressure. For example, the sensors 906 and 908 may be pressure sensors configured to detect mechanical pressure on the sensors 906 and 908. As another example, the sensors 906 and 908 may be light sensors configured to detect reduction in light incident on the sensors 906 and 908 due to placement of the user's fingers on the sensors 906 and 908 when applying mechanical pressure. As another example, the sensors 906 and 908 may be temperature sensors configured to detect an increase in temperature of the sensors 906 and 908 due to placement of the user's fingers on the sensors 906 and 908 when applying mechanical pressure. In some embodiments, upon detection of the application of mechanical pressure by the sensors 906 and 908, processing circuitry may be configured to trigger the ultrasound-on-a-chip device 110 in the ultrasound module 104 to collect ultrasound data. The processing circuitry may be processing circuitry 112 or processing circuitry in an external host device (e.g., a smartphone, tablet, or laptop), workstation, or server.

In some embodiments, the coupling strip 148 may be configured such that it does not establish acoustic coupling between the ultrasound-on-a-chip device 110 and the user's wrist during normal use. In such embodiments, the user may apply light mechanical pressure to the recesses 902 and 904, and the mechanical pressure may cause the coupling strip 148 opposite the recesses 902 and 904 to establish acoustic coupling between the ultrasound-on-a-chip device 110 and the user's wrist. Accordingly, when the user applies the mechanical pressure, the coupling strip 148 may establish acceptable coupling between the ultrasound module 104 and the user's wrist and enable the ultrasound-on-a-chip device 110 to collect ultrasound data of acceptable quality. In some embodiments, the ultrasound-on-a-chip device 110 may collect ultrasound data (continuously or at intervals), and processing circuitry, such as processing circuitry 112 or processing circuitry in an external host device (e.g., a smartphone, tablet, or laptop), workstation, or server, may later analyze the collected ultrasound data to detect ultrasound data collected when the mechanical pressure was not applied and delete that ultrasound data. In some embodiments, the ultrasound-on-a-chip device 110 may collect ultrasound data (continuously or at intervals), and the processing circuitry may later analyze the collected ultrasound data to detect ultrasound data collected when the mechanical pressure was applied and transmit that ultrasound data for storage in memory (e.g., memory circuitry 114, or memory in an external host device, workstation, or server). In such embodiments, to detect ultrasound data collected when mechanical pressure was applied and when mechanical pressure was not applied, the processing circuitry may calculate a measure of quality of the ultrasound data. In this embodiment, the sensors 906 and 908 may be absent. In some embodiments, data from the sensors 906 and 908 may be temporally correlated with ultrasound data (e.g., through the use of timestamps), and the processing circuitry may determine from the sensor data which ultrasound data was collected during periods with and without application of mechanical pressure. It may be helpful for the coupling strip 148 to not establish acoustic coupling between the ultrasound-on-a-chip device 110 and the user's wrist during normal use to avoid discomfort (e.g., due to constant rubbing of the coupling strip 148 against the user's wrist and difficulty in removing the coupling strip 148 from the user's wrist due to adhesion).

The recesses 902 and 904 may be incorporated into any of the apparatuses discussed herein. In some embodiments, the recesses 902 and 904 may not be located on the ultrasound-on-a-chip device 110. For example, the recesses 902 and 904 may be incorporated into the primary module 102 or into the first wristband 106. In some embodiments, there may be one recess or more than two recesses, the recesses 902 and 904 may be absent, and/or the sensors 906 and 908 may not be located in the recesses 902 and 904 and may detect the application of mechanical pressure by detecting signals (e.g., pressure signals) elsewhere in the apparatus. In some embodiments, applying mechanical pressure to the recesses 902 and 904 may compress reservoirs (e.g., reservoirs 802 and 804) within the ultrasound module 104 and cause them to expel liquid or gel to refresh the coupling strip 148.

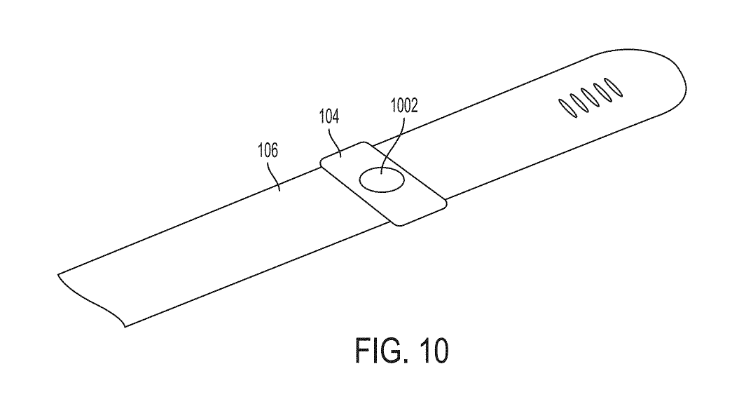

FIG. 10 shows an example of a mechanical button incorporated into an ultrasound module in accordance with certain embodiments disclosed herein. FIG. 10 shows the ultrasound module 104 and the first wristband 106. The ultrasound module 104 is coupled to the first wristband 106 and incorporates a mechanical button 1002 on the outer surface of the ultrasound module 104 (i.e., the surface that faces away from the user's wrist). In some embodiments, upon detection of activation of the mechanical button 1002 (e.g., application of mechanical pressure to the mechanical button 1002), processing circuitry, such as processing circuitry 112 or processing circuitry in an external host device (e.g., a smartphone, tablet, or laptop), workstation, or server, may be configured to trigger the ultrasound-on-a-chip device 110 to collect ultrasound data. In some embodiments, applying mechanical pressure to the mechanical button 1002 may trigger reservoirs (e.g., reservoirs 802 and 804) within the ultrasound module 104 to release liquid or gel to refresh the coupling strip 148. In some embodiments, there may be more than one mechanical button 1002, or the mechanical button 1002 may be located on the first wristband 106.

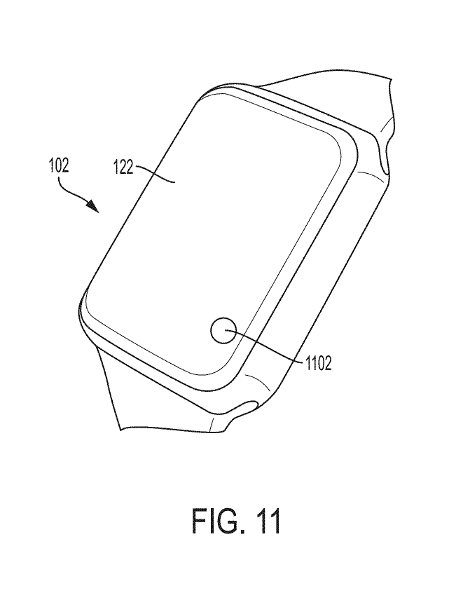

FIG. 11 shows an example of a virtual button on a display screen of a primary module in accordance with certain embodiments disclosed herein. FIG. 11 shows the primary module 102. The primary module 102 includes the display screen 122 which displays a virtual button 1102. In some embodiments, upon detecting that the virtual button 1102 has been activated (e.g., touched), processing circuitry, such as processing circuitry 112 or processing circuitry in an external host device (e.g., a smartphone, tablet, or laptop), workstation, or server, may be configured to trigger the ultrasound-on-a-chip device 110 to trigger the ultrasound-on-a-chip device 110 to collect ultrasound data. In some embodiments, activating the virtual button 1102 may cause the processing circuitry to trigger reservoirs (e.g., reservoirs 802 and 804) within the ultrasound module 104 to release liquid or gel to refresh the coupling strip 148. The virtual button 1102 may be located on any portion of the display screen 122.

FIG. 12 shows an example of a mechanical button on a primary module in accordance with certain embodiments disclosed herein. FIG. 12 shows the primary module 102 which includes a mechanical button 1202 in a side wall of the primary module 102. In some embodiments, upon detecting that the mechanical button 1202 has been activated (e.g., pressed), processing circuitry, such as processing circuitry 112 or processing circuitry in an external host device (e.g., a smartphone, tablet, or laptop), workstation, or server, may be configured to trigger the ultrasound-on-a-chip device 110 to trigger the ultrasound-on-a-chip device 110 to collect ultrasound data. In some embodiments, activating the mechanical button 1202 may cause the processing circuitry to trigger reservoirs (e.g., reservoirs 802 and 804) within the ultrasound module 104 to release liquid or gel to refresh the coupling strip 148. The mechanical button 1202 may be located on any portion of the primary module. One or more of the recesses 902 and 904, mechanical button 1002, virtual button 1102, and mechanical button 1202 may all be included in the apparatus.

### Example Data Collection and Processing

In some embodiments, processing circuitry may be configured to process and/or analyze ultrasound images (which may be two-dimensional images or, when the ultrasound-on-a-chip device 110 includes a two-dimensional array, three-dimensional images) reconstructed from ultrasound data collected by the ultrasound-on-a-chip device 110. In some embodiments, the processing circuitry may be configured to analyze the ultrasound data itself. The processing circuitry may be processing circuitry 112 or processing circuitry in an external host device (e.g., a smartphone, tablet, or laptop), workstation, or server. The processing circuitry may trigger ultrasound data collection by the ultrasound-on-a-chip device 110 and/or analyze ultrasound data/images at a single time, or at time intervals (e.g., every second, every minute, every hour, four times per day, three times per day, two times per day, one a day, or any suitable time interval), or continuously. In some embodiments, the processing circuitry may use deep learning models to analyze ultrasound data/images. In such embodiments, the processing circuitry may be configured to retrieve, from a server, ultrasound data/images from other ultrasound-on-a-chip devices and to use the ultrasound data/images from the other ultrasound-on-a-chip devices when training the deep learning models. For further discussion of deep learning models, see U.S. patent application Ser. No. 15/626,423 titled “AUTOMATIC IMAGE ACQUISITION FOR ASSISTING A USER TO OPERATE AN ULTRASOUND DEVICE.”.

In some embodiments, the processing circuitry may reconstruct ultrasound data collected from the wrist to form an ultrasound image (which may be two-dimensional images or, when the ultrasound-on-a-chip device 110 includes a two-dimensional array, three-dimensional images). The processing circuitry may analyze the ultrasound image to perform segmentation of the ultrasound image to identify contours of anatomical structures displayed in the ultrasound image, such as blood vessels within the wrist (e.g., the radial artery, ulnar artery, and median artery). The processing circuitry may perform various anatomical and physiological measurements using the ultrasound image, such as measuring the diameter of a blood vessel displayed in the ultrasound image; measuring the average, minimum, and/or maximum of the diameter over time of a blood vessel displayed in the ultrasound image; measuring blood pressure; measuring velocity of blood flow within a blood vessel displayed in the ultrasound image; producing a map of velocity of blood flow within a blood vessel; producing a time trace of heart rate; and producing a time trace of velocity of blood flow within a blood vessel displayed in the ultrasound image.

In some embodiments, the ultrasound-on-a-chip device 110 may be configured to perform Doppler ultrasound imaging, which may include pulsed wave Doppler imaging. The processing circuitry may be configured to form a color Doppler ultrasound image based on ultrasound data collected with pulsed wave Doppler imaging, and may also be configured to form a spectral Doppler velocity trace of blood flow within a blood vessel based on ultrasound data. In some embodiments, the processing circuitry may be configured to measure average velocity, maximum velocity, minimum velocity, and/or acceleration of blood flow within a blood vessel based on ultrasound data collected with Doppler ultrasound imaging over a period of time. In some embodiments, the processing circuitry may be configured to measure blood volume flowing within a blood vessel per heart pulse. In some embodiments, the processing circuitry may be configured to produce a time trace based on M-mode ultrasound imaging.

In some embodiments, the ultrasound-on-a-chip device 110 may be configured to perform ultrasound elastography (e.g., shear wave elasticity imaging, quasistatic elastography, acoustic radiation force impulse imaging, shear imaging, and transient elastography) and the processing circuitry may be configured to produce data and/or form an image based on the ultrasound data collected. In some embodiments, the data produced from the ultrasound elastography may include measurements of elasticity of a blood vessel wall. In some embodiments, measurement of elasticity of a blood vessel wall may be combined with measurement of blood volume within the blood vessel to calculate blood pressure.

In some embodiments, the processing circuitry may be configured to calculate pulse wave velocity (PWV) in a blood vessel based on ultrasound data from the blood vessel. PWV may represent a measure of arterial stiffness, which in some cases has been proven to be a predictor of cardiovascular disease. PWV may represent a noninvasive method for measuring arterial blood pressure waveforms, which may contain information for diagnosing and treating cardiovascular disease. In some embodiments, the processing circuitry may be configured to calculate PWV in the blood vessel periodically over time and to output the evolution of PWV calculations over time.

In some embodiments, the processing circuitry may calculate PWV at a single arterial site based on volumetric flow rate and cross-sectional area measured at the arterial site. The arterial site may be a site along on an artery in the wrist, such as the radial artery, ulnar artery, and median artery. In such embodiments, the processing circuitry may calculate cross-sectional area by receiving, from the ultrasound-on-a-chip device 110 positioned at the wrist, data from a transverse ultrasound scan of the blood vessel in the wrist, measuring the diameter of the blood vessel on an ultrasound image resulting from the transverse scan, and calculating the cross-sectional area from the measured diameter by assuming axisymmetrical geometry. The processing circuitry may calculate volumetric flow rate by multiplying cross-sectional area by spatial mean velocity. The processing circuitry may calculate spatial mean velocity by receiving, from the ultrasound-on-a-chip device 110 positioned at the wrist, pulsed Doppler ultrasound imaging of the blood vessel in the wrist, where the blood vessel is insonated at an angle relative to the longitudinal axis of the blood vessel. The angle may be, for example, <10 degrees, 10 degrees, 20 degrees, 30 degrees, 40 degrees, 50 degrees, 70 degrees, 80 degrees, >80 degrees, or any other suitable angle. In some embodiments, to perform the pulsed Doppler ultrasound imaging at an angle relative to the longitudinal axis of the blood vessel, the ultrasound-on-a-chip device 110 may perform a transverse ultrasound scan with an elevational steer towards the blood vessel. In some embodiments, to perform the pulsed Doppler ultrasound imaging at an angle relative to the longitudinal axis of the blood vessel, the ultrasound-on-a-chip device 110 may perform a longitudinal ultrasound scan steered at an angle towards the blood vessel. PWV may be calculated from the volumetric flow rate and cross-sectional area measurements by measuring the slope of a linear portion of a volumetric flow rate vs. cross-sectional area plot. Furthermore, blood pressure may be estimated based on PWV and the cross-sectional area of the blood vessel. As discussed above, PWV is a measure of arterial stiffness, or conversely, arterial elasticity, and thus other methods for measuring elasticity using ultrasound imaging may be used to estimate blood pressure. Other methods for measuring elasticity using ultrasound may include, for example, shear wave elasticity imaging, quasistatic elastography, acoustic radiation force impulse imaging, shear imaging, and transient elastography. For further description of measuring PWV based on volumetric flow rate and cross-sectional area measured at an arterial site, and estimating blood pressure using PWV/elasticity, see Seo, Joohyun, et al. “Noninvasive arterial blood pressure waveform monitoring using two-element ultrasound system.” IEEE transactions on ultrasonics, ferroelectrics, and frequency control 62.4 (2015): 776-784, which is hereby incorporated by reference herein in its entirety. In some embodiments, the ultrasound-on-a-chip device 110 may perform a bi-planar acquisition, where the diameter of the blood vessel is measured using transverse scanning and the spatial mean velocity is measured using longitudinal scanning. When the ultrasound-on-a-chip device 110 includes a two-dimensional array of ultrasound transducers, the ultrasound-on-a-chip device 110 may be configured to perform both transverse ultrasound scanning and longitudinal ultrasound scanning, with or without azimuthal and/or elevational steering, without requiring rotation of the ultrasound-on-a-chip device 110 relative to the user's wrist.

In some embodiments, the processing circuitry may calculate PWV by receiving, from the ultrasound-on-a-chip device 110 positioned at the wrist, pulse wave imaging of blood vessel. For example, the blood vessel may be an artery in the wrist, such as the radial artery, ulnar artery, and median artery, or a vein in the wrist. In such embodiments, the processing circuitry may calculate cross-sectional area by receiving, from the ultrasound-on-a-chip device 110 positioned at the wrist, longitudinal ultrasound scanning at an acceptably high frame rate for detecting a pulse wave. The frame rate may be, for example, 500 Hz, 1000 Hz, 1500 Hz, 2000 Hz, or any suitable frame rate. The processing circuitry may analyze interframe axial displacements of the blood vessel to obtain velocities of the blood vessel wall, and PWV can be calculated based on the obtained velocities. For further description of measuring PWV using pulse wave imaging, see Luo, Jianwen, Ronny X. Li, and Elisa E. Konofagou. “Pulse wave imaging of the human carotid artery: an in vivo feasibility study.” IEEE transactions on ultrasonics, ferroelectrics, and frequency control 59.1 (2012): 132-181, which is hereby incorporated by reference herein in its entirety.

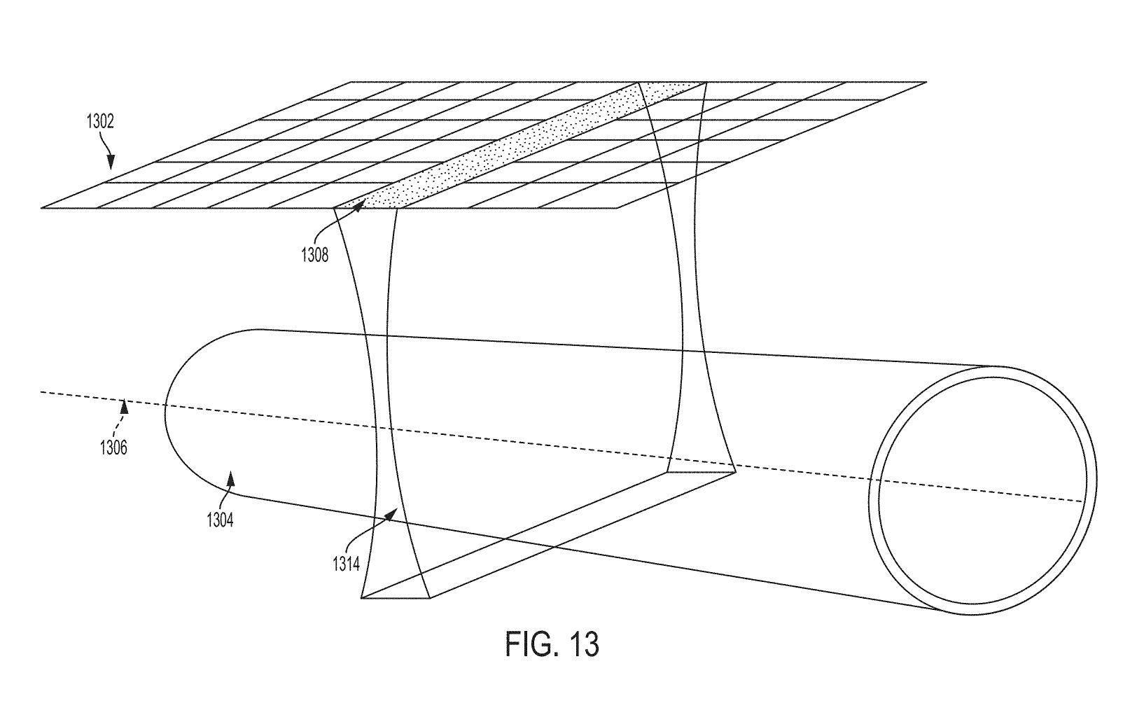

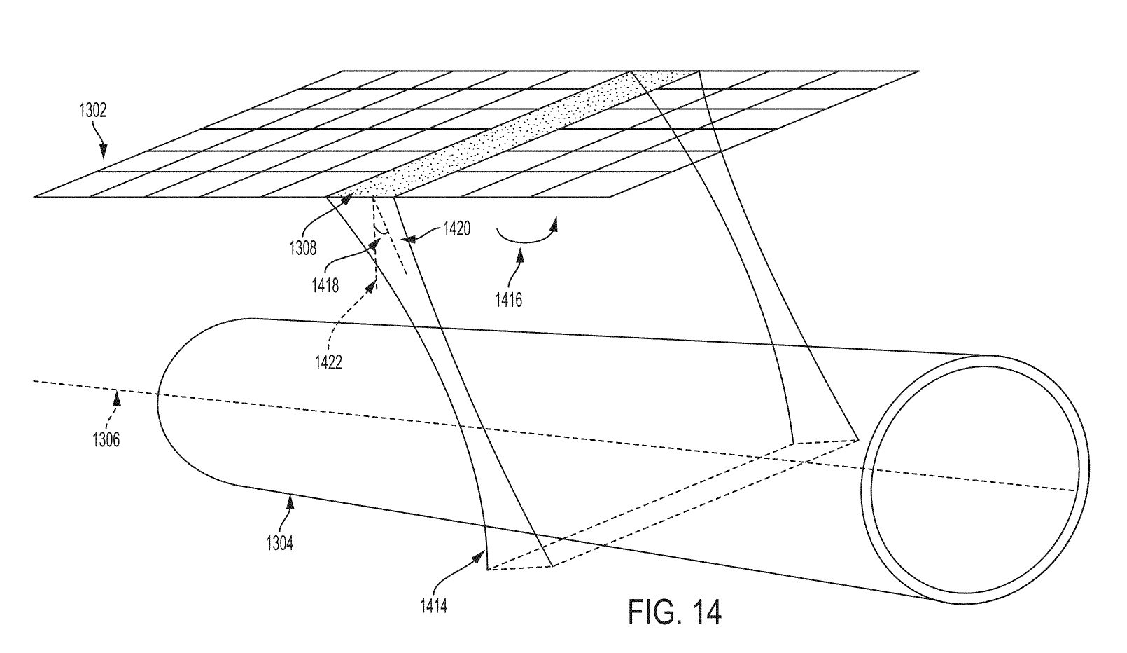

FIG. 13 shows an illustration of performing a transverse ultrasound scan of a blood vessel 1304 with a two-dimensional array of ultrasound transducers 1302 in accordance with certain embodiments disclosed herein. The blood vessel 1304 has a longitudinal axis 1306. The two-dimensional array of ultrasound transducers 1302 includes a set of ultrasound transducers 1308. In FIG. 13, the set of ultrasound transducers 1308 represents a column of the two-dimensional array of ultrasound transducers 1302. The set of ultrasound transducers 1308 is configured to perform a transverse ultrasound scan of the blood vessel 1304 by producing an ultrasound beam profile 1314 along a plane that is orthogonal to the longitudinal axis 1306 of the blood vessel 1304. It will be appreciated that the ultrasound beam profile 1314 (and the ultrasound beam profiles discussed in FIGS. 14-17) is a conceptual representation, and is shown as an approximation of multiple (e.g., up to hundreds of) transmit events that may overlap in space and may progressively advance in one direction and are used to form a cross-sectional ultrasound image slice. Thus, the ultrasound beam profile 1314 need not represent spatial locations for a single beam, but may display total spatial illumination that may be used to form a cross-sectional ultrasound image slice. The ultrasound beam profile 1314 also shows approximate intensity threshold bounds. By performing the transverse ultrasound scan of the blood vessel 1304, the set of ultrasound transducers 1308 may collect ultrasound data from which the diameter of the blood vessel 1304 can be measured. As discussed above, measuring the diameter of the blood vessel 1304 may be useful, for example, for calculating the cross-sectional area of the blood vessel 1304, which in turn can be useful for calculating PWV.

FIG. 14 shows an illustration of performing a transverse ultrasound scan, with an elevational steer, of the blood vessel 1304 using the two-dimensional array of ultrasound transducers 1302 in accordance with certain embodiments disclosed herein. The set of ultrasound transducers 1308 is configured to perform a transverse ultrasound scan, steered in the elevation direction, of the blood vessel 1304 by producing an ultrasound beam profile 1414 along a plane that is orthogonal to the longitudinal axis 1306 of the blood vessel 1304, similar to the ultrasound beam profile 1314. The set of ultrasound transducers 1308 is further configured to steer the ultrasound beam profile 1414 in an elevation direction 1416 such that the ultrasound beam profile 1414 forms an angle 1418 with a plane orthogonal to the longitudinal axis 1306 of the blood vessel 1304. (The angle 1418 is shown between a line 1420 parallel to a plane through the ultrasound beam profile 1414 and a line 1422 that is orthogonal to the longitudinal axis 1306). Performing the transverse ultrasound scan, steered in the elevation direction, of the blood vessel 1304 may be useful, for example, for performing pulsed Doppler ultrasound imaging of the blood vessel 1304, with which the spatial mean velocity of blood flow through the blood vessel 1304 may be measured. As discussed above, measuring the spatial mean velocity of blood flow through the blood vessel 1304 may be useful, for example, for calculating PWV.

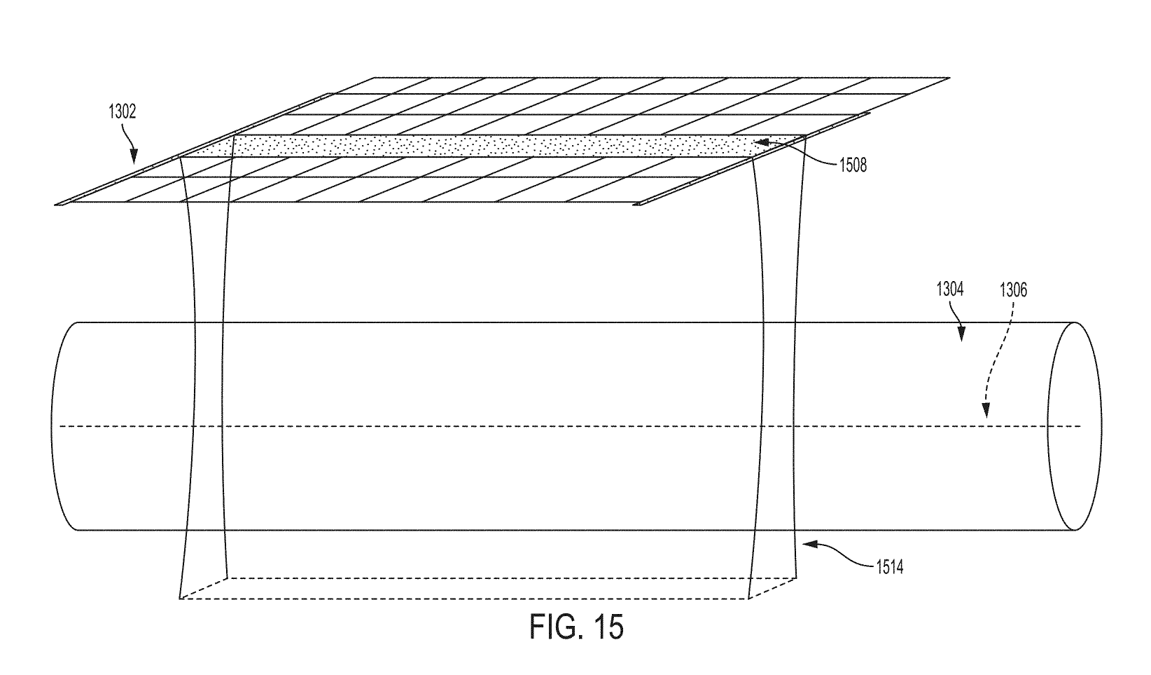

FIG. 15 shows an illustration of performing a longitudinal ultrasound scan of a blood vessel 1304 with the two-dimensional array of ultrasound transducers 1302 in accordance with certain embodiments disclosed herein. The two-dimensional array of ultrasound transducers 1302 includes a set of ultrasound transducers 1408. In FIG. 15, the set of ultrasound transducers 1508 represents a row of the two-dimensional array of ultrasound transducers 1302. The set of ultrasound transducers 1508 is configured to perform a longitudinal ultrasound scan of the blood vessel 1304 by producing an ultrasound beam profile 1514 along a plane that is parallel to the longitudinal axis 1306 of the blood vessel 1304. By performing the longitudinal ultrasound scan of the blood vessel 1304, the set of ultrasound transducers 1508 may be able to perform pulse wave imaging. As discussed above, performing pulse wave imaging may be useful, for example, for calculating blood vessel velocities, which can in turn be useful for calculating PWV.

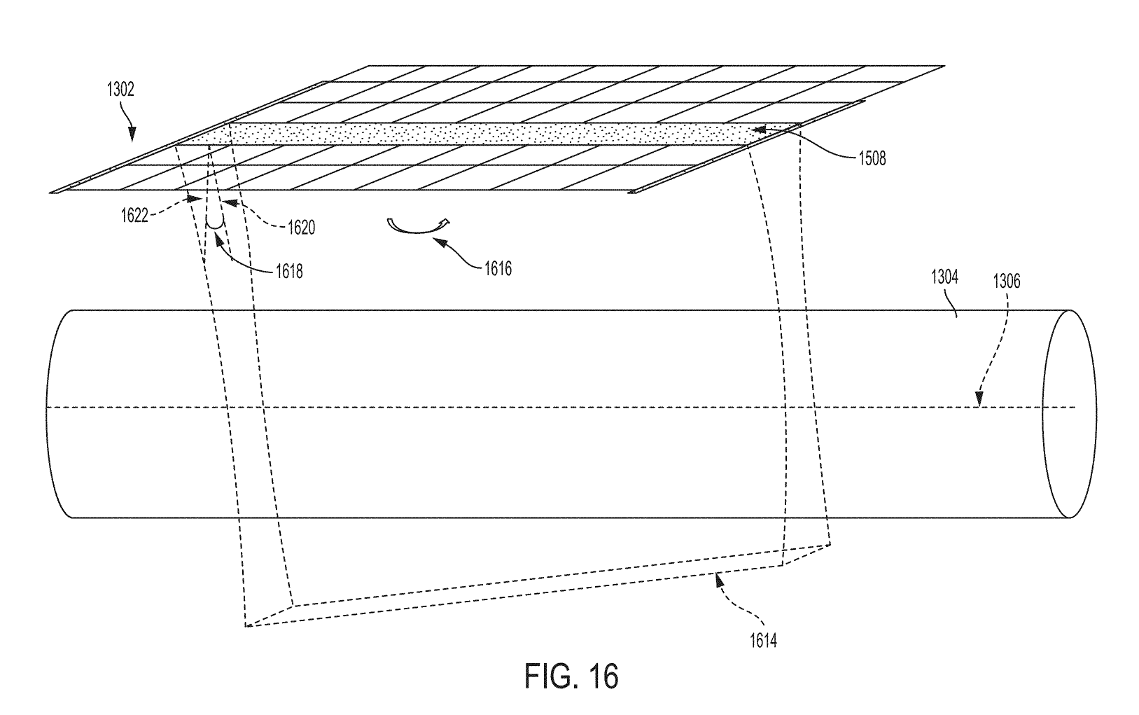

FIG. 16 shows an illustration of performing a longitudinal ultrasound scan, with an azimuthal steer, of a blood vessel 1304 using the two-dimensional array of ultrasound transducers 1302 in accordance with certain embodiments disclosed herein. The set of ultrasound transducers 1508 is configured to perform a longitudinal ultrasound scan of the blood vessel 1304 by producing an ultrasound beam profile 1614 along a plane that is parallel to the longitudinal axis 1306 of the blood vessel 1304, similar to the ultrasound beam profile 1514. The set of ultrasound transducers 1508 is further configured to steer the ultrasound beam profile 1614 in an azimuthal direction 1616 such that the ultrasound beam profile forms an angle 1618 with a plan that is orthogonal to the longitudinal axis 1306 of the blood vessel 1304. (The angle 1618 is shown between a line 1620 parallel to a plane through the ultrasound beam profile 1614 and a line 1622 that is orthogonal to the longitudinal axis 1306). Performing the longitudinal ultrasound scan of the blood vessel 1304 with an azimuthal steer may be useful, for example, for performing pulsed Doppler ultrasound imaging of the blood vessel 1304, with which the spatial mean velocity of blood flow through the blood vessel 1304 may be measured. As discussed above, measuring the spatial mean velocity of blood flow through the blood vessel 1304 may be useful, for example, for calculating PWV.

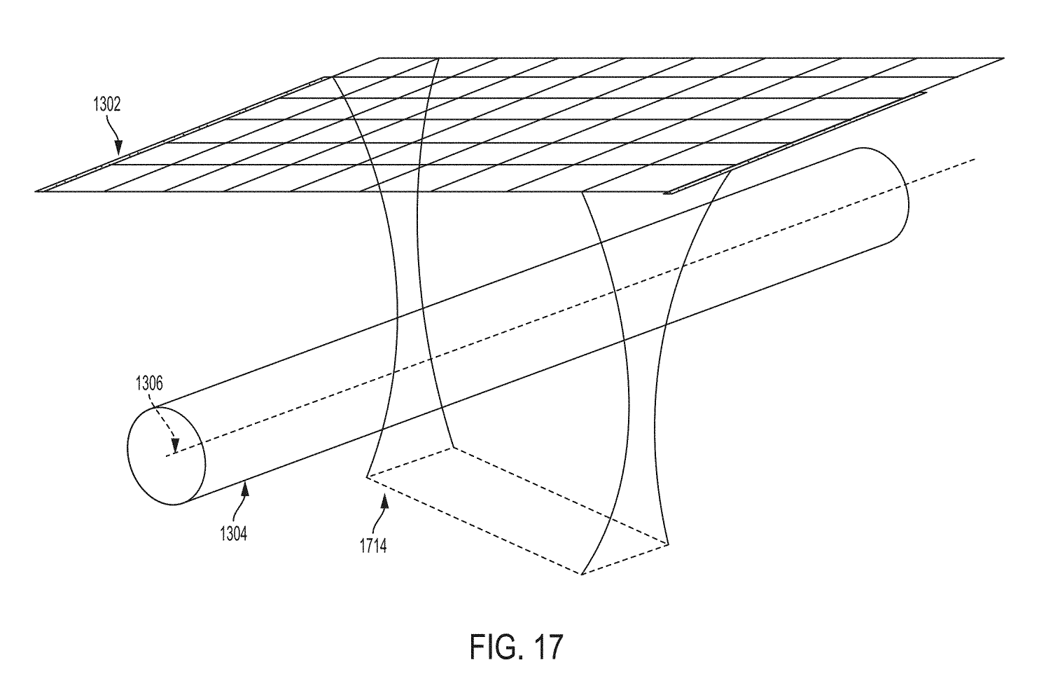

FIG. 17 shows an illustration of performing a transverse ultrasound scan of a blood vessel 1304 using the two-dimensional array of ultrasound transducers 1302, when the blood vessel does not lie either perpendicular or parallel to the azimuth direction or the elevation direction, in accordance with certain embodiments disclosed herein. In FIG. 17, all of the transducers in the two-dimensional array of ultrasound transducers 1302 are used to produce an ultrasound beam profile 1714 along a plane that is perpendicular to the longitudinal axis 1306 of the blood vessel 1304. However, in some embodiments, a subset of the transducers in the two-dimensional array of ultrasound transducers 1302 are used. While a transverse ultrasound scan is shown in FIG. 17, the two-dimensional array of ultrasound transducers 1302 may be configured to perform a longitudinal ultrasound scan, or steer an ultrasound beam profile in any arbitrary direction, even when the blood vessel does not lie perpendicular or parallel to the azimuth direction or the elevation direction. Thus, the two-dimensional array of ultrasound transducers 1302 can perform a transverse or longitudinal scan on the blood vessel 1304 without rotating the two-dimensional array of ultrasound transducers 1302, as the scanning direction can be rotated about the normal axis of two-dimensional array of ultrasound transducers 1302 such that a transverse scan or longitudinal scan can be performed with any relative orientations between the two-dimensional array of ultrasound transducers 1302 and the blood vessel 1304. This may be helpful, for example, if the wrist bound ultrasound-on-a-chip device 110 moves during normal use and does not maintain a constant orientation relative to the blood vessel 1304.

In the examples of FIGS. 13-17, the two-dimensional array of ultrasound transducers 1302 may be nonuniform (e.g., the transducers may be arranged in a configuration that is not a regular rectangular grid) in some embodiments. In some embodiments, more than one column of transducers may be configured to produce the ultrasound beam profiles, or any group of transducers in the two-dimensional array of ultrasound transducers 1302 (e.g., all the transducers) may be configured to produce the ultrasound beam profiles. In some embodiments, the ultrasound beam profiles may be formed from cylindrical beams and/or plane waves, and the ultrasound beam profiles may have sector profiles and may be formed from focused beams.

As illustrated by FIGS. 13-17, the two-dimensional array of ultrasound transducers 1302 may be helpful in a wrist bound ultrasound-on-a-chip device, as the two-dimensional array of ultrasound transducers 1302 can, for example, perform transverse and longitudinal ultrasound scanning and steer ultrasound beam profiles in the azimuth and elevational directions, or rotate/steer ultrasound beam profiles in any other arbitrary direction, without requiring rotation of the ultrasound-on-a-chip device relative to the user's wrist between scans. In some embodiments, the two-dimensional array of ultrasound transducers 1302 may use a set of transducers (e.g., set of ultrasound transducers 1308) in a first direction to form ultrasound beam profiles in a traverse direction (e.g., ultrasound beam profiles 1314 and 1414) and another set of transducers (e.g., set of ultrasound transducers 1508) in a second direction that is orthogonal to the first direction to form ultrasound beam profiles in a longitudinal direction (e.g., ultrasound beam profiles 1514 and 1614). This flexibility can be useful, for example, in applications requiring collection of multiple types of data that may require or be enabled by multiple ultrasound beam profiles and multiple scanning directions. For example, measuring PWV at the wrist may require collecting data for measuring blood vessel diameter, spatial mean velocity, and/or blood vessel wall velocity, which may be enabled by the flexibility of a two-dimensional ultrasound transducer array.

### Example System Functions

Processing circuitry may be configured to perform functions related to the apparatuses described herein. The processing circuitry may be processing circuitry 112 or processing circuitry in an external host device (e.g., a smartphone, tablet, or laptop), workstation, or one or more servers (also known as a “cloud”). In some embodiments, the processing circuitry may be configured to detect when the ultrasound-on-a-chip device 110 needs to be repositioned. In some embodiments, detecting when the ultrasound-on-a-chip device 110 needs to be repositioned includes determining whether a current deviation of the ultrasound-on-a-chip device 110 from a desired position exceeds a threshold deviation. For example, the ultrasound-on-a-chip device 110 may be positioned over a specific portion of the wrist from which an ultrasound image containing a target anatomical view may be collected, but due to movement of the user's wrist, the ultrasound-on-a-chip device 110 may move away from the specific portion of the wrist. In some embodiments, to detect when the ultrasound-on-a-chip device 110 needs to be repositioned, the processing circuitry may be configured to analyze (continuously or at intervals) data collected by the ultrasound-on-a-chip device and determine whether the collected data matches the desired collected data. For example, the processing circuitry may collect ultrasound data, form an ultrasound image, and determine whether the ultrasound image contains a target anatomical view (e.g., a view of a specific anatomical structure, such as a specific blood vessel). In some embodiments, the processing circuitry may use deep learning to detect when the ultrasound-on-a-chip device needs to be repositioned.

In some embodiments, the processing circuitry may be configured to generate a notification to reposition the ultrasound-on-a-chip device 110. The notification may include instructions to the user to move the ultrasound-on-a-chip device 110 to the required position, and the instructions may guide the user in moving the ultrasound-on-a-chip device 110 to the required position. For further description of guiding a user in moving an ultrasound-on-a-chip device 110 to a required position, see U.S. patent application Ser. No. 15/626,423 titled “AUTOMATIC IMAGE ACQUISITION FOR ASSISTING A USER TO OPERATE AN ULTRASOUND DEVICE.” In some embodiments, the notification may be displayed on the display screen 122, and may include text, an image, and/or a video. In some embodiments, the notification may include audio output from the primary module 102.

In some embodiments, the processing circuitry may be configured to detect when the coupling strip 148 needs to be refreshed with liquid or gel or replaced. In some embodiments, detecting that the coupling strip 148 needs to be refreshed with liquid or gel includes determining whether a current amount of liquid or gel associated with the coupling strip 148 is below a threshold amount. For example, the liquid or gel absorbed in the coupling strip 148 may evaporate, which can cause the coupling strip 148 to conform poorly to the user's wrist, and thereby cause the ultrasound-on-a-chip device 110 to collect poor quality ultrasound images. In some embodiments, the processing circuitry may be configured to analyze collected data (continuously or at intervals) and determine whether the collected data shows signs (e.g., decreased quality of images) that the coupling strip 148 is conforming poorly to the user's wrist. To detect that the coupling strip 148 needs to be refreshed with liquid or gel or replaced, the processing circuitry may be configured to receive signals from a moisture sensor in or adjacent to the coupling strip 148 indicating that the moisture level in or adjacent to the coupling strip 148 is below a threshold moisture level. In some embodiments, the processing circuitry may be configured to use other sensors to detect that the coupling strip 148 needs to be refreshed, such as capacitive sensors or skin conductivity sensors. In some embodiments, the processing circuitry may use deep learning to detect when the coupling strip needs to be refreshed or replaced.

In some embodiments, the processing circuitry may be configured to generate a notification to replace the coupling strip 148 or refresh the coupling strip 148 with liquid or gel. The notification may include instructions to the user to refresh the coupling strip 148. The notification may be displayed on the display screen 122 and may include text, an image, and/or a video. In some embodiments, the notification may include audio output from the primary module 102.

In some embodiments, the processing circuitry may be configured to generate for display data and/or images generated based on ultrasound data collected from the user's wrist. The processing circuitry may generate for display the data/images on the display screen 122 or on a display screen of an external host device (e.g., smartphone, tablet, or laptop), workstation, or server. In some embodiments, the user may access a software application (“app”) installed on a host device or workstation for viewing the data/images. In some embodiments, the processing circuitry may receive the data/images from a remote server.

### Example Processes

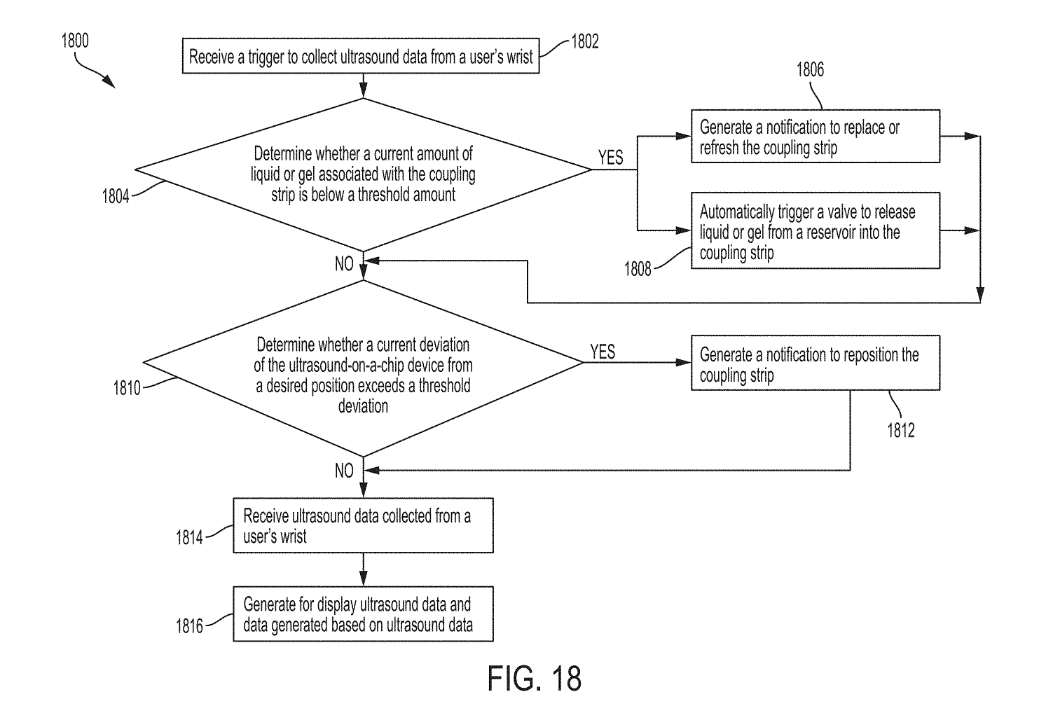

FIG. 18 shows an example process 1800 for obtaining ultrasound data from a user's wrist, in accordance with certain embodiments disclosed herein. The ultrasound data may be received from the ultrasound-on-chip device 110 in any of the apparatuses described herein. The process 1800 may be performed by, for example, processing circuitry. The processing circuitry may be processing circuitry 112 or processing circuitry in an external host device (e.g., a smartphone, tablet, or laptop), workstation, or server.

In act 1802, the processing circuitry may receive a trigger to collect ultrasound data from a user's wrist. In some embodiments, the trigger may be passage of a fixed period of time (e.g., one second, one minute, one hour, 6 hours, 8 hours, 12 hours, one day, or any suitable period of time). In some embodiments, the trigger may be activation of a button (e.g., virtual button 1102 or mechanical button 1202) or a sensor (e.g., sensors 924 and 925) on the apparatus. The process 1800 may then proceed to act 1804.

In act 1804, the processing circuitry may determine whether a current amount of liquid or gel associated with the coupling strip 148 is below a threshold amount. In some embodiments, to determine whether the current amount of liquid or gel associated with the coupling strip 148 is below the threshold amount, the processing circuitry may be configured to analyze (continuously or periodically) ultrasound data collected by the ultrasound-on-a-chip device 110 and determine whether the collected ultrasound data shows signs (e.g., decreased quality of images) that the coupling strip 148 is conforming poorly to the user's wrist. In some embodiments, to determine whether the current amount of liquid or gel associated with the coupling strip 148 is below the threshold amount, the processing circuitry may be configured to receive signals from a moisture sensor in or adjacent to the coupling strip 148 indicating that the moisture level in or adjacent to the coupling strip 148 is below a threshold moisture level. In some embodiments, the processing circuitry may be configured to use other sensors to determine whether the current amount of liquid or gel associated with the coupling strip 148 is below the threshold amount, such as capacitive sensors or skin conductivity sensors. If the current amount of liquid or gel associated with the coupling strip 148 is not below the threshold amount, the process 1800 may proceed to act 1810. If the current amount of liquid or gel associated with the coupling strip 148 is below the threshold amount, the process 1800 may proceed to act 1806 and/or to act 1808. Processor-executable instructions (e.g., stored in memory circuitry 114) may determine whether the process 1800 proceeds to act 1806 and/or to act 1808.

In act 1806, the processing circuitry may generate a notification to replace or refresh the coupling strip 148. In some embodiments, the notification may be displayed on the display screen 122, or on a display screen of an external host device (e.g., a smartphone, tablet, or a computer) local to the user, and may include text, an image, and/or a video. In some embodiments, the notification may include audio output from the primary module 102 or from an external host device. The process may then proceed to act 1810.

In act 1808, the processing circuitry may automatically trigger a valve (e.g., valves 808 and 812) to release liquid or gel from a reservoir (e.g., reservoirs 802 and 804) into the coupling strip 148. The process 1800 may then proceed to act 1810.

In act 1810, the processing circuitry may determine whether a current deviation of the ultrasound-on-a-chip device 110 from a desired position exceeds a threshold deviation. In some embodiments, the processing circuitry may analyze collected data (continuously or at intervals) and determine whether the collected data matches the desired collected data. For example, the processing circuitry may receive ultrasound data, form an ultrasound image, and determine whether the ultrasound image contains a target anatomical view (e.g., a view of a specific anatomical structure, such as a specific blood vessel). If the current deviation of the ultrasound-on-a-chip device 110 from the desired position does not exceed the threshold deviation, the process 1800 may proceed to act 1814. If the current deviation of the ultrasound-on-a-chip device 110 from the desired position exceeds the threshold deviation, the process 1800 may proceed to act 1812.

In act 1812, the processing circuitry may generate a notification to reposition the ultrasound-on-a-chip device 110. In some embodiments, the notification may include instructions to the user to move the ultrasound-on-a-chip device 110 to the required position, and may include instructions that guide the user in moving the ultrasound-on-a-chip device to the required position. For further description of guiding a user in moving an ultrasound-on-a-chip device to a required position, see U.S. patent application Ser. No. 15/626,423 titled “AUTOMATIC IMAGE ACQUISITION FOR ASSISTING A USER TO OPERATE AN ULTRASOUND DEVICE.” In some embodiments, the notification may be displayed on the display screen 122 or a display screen of an external host device (e.g., a smartphone, tablet, or a computer) local to the user, and may include text, an image, and/or a video. In some embodiments, the notification may include audio output from the primary module 102 or an external host device local to the user. The process may then proceed to act 1814.

In act 1814, the processing circuitry may receive ultrasound data collected from the user's wrist. In some embodiments in which the processing circuitry is external to the ultrasound-on-a-chip device 110, the processing circuitry may receive the ultrasound data over conductors 136/connection cable 376 or over a wireless communication link such as a BLUETOOTH, WiFi, or ZIGBEE wireless communication link using communication circuitry (e.g., communication circuitry 116). The process may then proceed to act 1816.

In act 1816, the processing circuitry may generate for display the ultrasound data, an ultrasound image generated based on the ultrasound data, and/or data generated based on the ultrasound data. For example, the data generated based on the ultrasound data may include calculations of blood flow, heart rate, blood pressure, blood vessel diameter, and pulse wave velocity based on the ultrasound data. The processing circuitry may generate for display the ultrasound data, ultrasound image, and/or data generated based on the ultrasound data on the display screen 122 or on a display screen of an external host device (e.g., a smartphone, a tablet, or a computer) local to the user. In some embodiments, the user may access a software application (“app”) installed on the primary module 102 or host device for viewing the data and/or images.

In some embodiments, certain steps in process 1800 may be omitted. For example, the process 1800 may not determine whether the current deviation of the ultrasound-on-a-chip device 110 from the desired position exceeds a threshold deviation, may not determine whether the current amount of liquid or gel associated with the coupling strip 148 is below the threshold amount, and/or may not generate for display the ultrasound data/data generated based on the ultrasound data. In some embodiments, certain steps may be performed in a different order than shown in FIG. 18. For example, the process 1800 may including determining whether the current deviation of the ultrasound-on-a-chip device 110 from the desired position exceeds a threshold deviation before determining whether the current amount of liquid or gel associated with the coupling strip 148 is below the threshold amount.

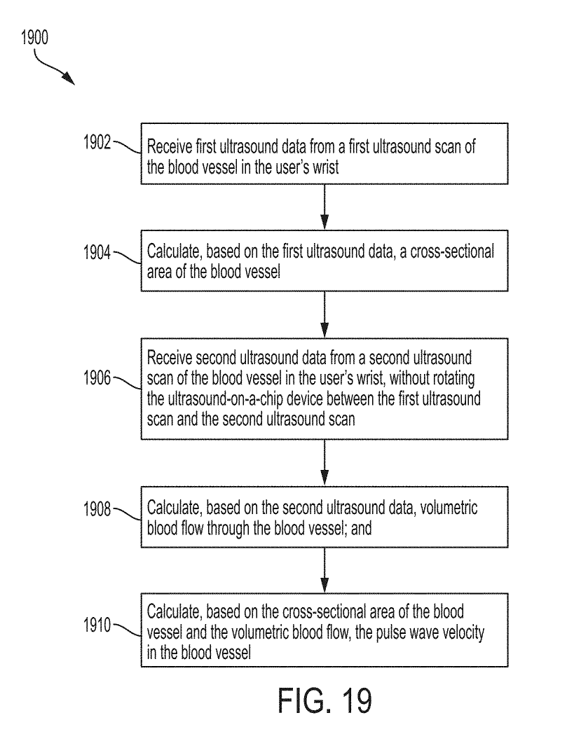

FIG. 19 shows an example process 1900 for calculating pulse wave velocity (PWV), in accordance with certain embodiments disclosed herein. PWV may represent a measure of arterial stiffness, which in some cases, such as aortic stiffness, has been proven to be a predictor of cardiovascular disease. PWV may represent a noninvasive method for measuring arterial blood pressure waveforms, which may contain information for diagnosing and treating cardiovascular disease. The ultrasound data may be received from the ultrasound-on-chip device 110 in any of the apparatuses described herein. The process 1900 may be performed by, for example, processing circuitry. The processing circuitry may be processing circuitry 112 or processing circuitry in an external host device (e.g., a smartphone, tablet, or laptop), workstation, or server. In some embodiments, the processing circuitry may calculate PWV at a single arterial site based on volumetric flow rate and cross-sectional area measured at the arterial site. The arterial site may be a site along on an artery in the wrist, such as the radial artery, ulnar artery, and median artery.

In act 1902, the processing circuitry may receive first ultrasound data from a first ultrasound scan of the blood vessel in the user's wrist. In some embodiments, the first ultrasound scan of the blood vessel may be a transverse ultrasound scan of the blood vessel. The process 1900 may then proceed to act 1904.

In act 1904, the processing circuitry may calculate, based on the first ultrasound data, a cross-sectional area of the blood vessel. In some embodiments, the processing circuitry may measure the diameter of the blood vessel on an ultrasound image resulting from a transverse ultrasound scan, and calculating the cross-sectional area from the measured diameter involves assuming axisymmetrical geometry. In some embodiments, processing circuitry may use deep learning to measure the diameter of the blood vessel based on the first ultrasound data. The process 1900 may then proceed to act 1906.

In act 1906, the processing circuitry may receive second ultrasound data from a second ultrasound scan of the blood vessel in the user's wrist, without rotating the ultrasound-on-a-chip device 110 relative to the user's wrist between the first ultrasound scan and the second ultrasound scan. In some embodiments, the second ultrasound scan may include performing pulsed Doppler ultrasound imaging of the blood vessel in the wrist, where the blood vessel is insonated at an angle relative to the longitudinal axis of the blood vessel. The angle may be, for example, <10 degrees, 10 degrees, 20 degrees, 30 degrees, 40 degrees, 50 degrees, 70 degrees, 80 degrees, >80 degrees, or any other suitable angle. In some embodiments, to perform the pulsed Doppler ultrasound imaging at an angle relative to the longitudinal axis of the blood vessel, the ultrasound-on-a-chip device may perform a transverse ultrasound scan with an elevational steer. In some embodiments, to perform the pulsed Doppler ultrasound imaging at an angle relative to the longitudinal axis of the blood vessel, the ultrasound-on-a-chip device may perform a longitudinal ultrasound scan steered at an angle. In some embodiments, to perform the second ultrasound scan without rotating the ultrasound-on-a-chip device relative to the user's wrist between the first ultrasound scan and the second ultrasound scan, the ultrasound-on-a-chip device 110 may include a two-dimensional array of ultrasound transducers. In some embodiments, the ultrasound-on-a-chip device 110 may use a one-dimensional array of ultrasound transducers to perform the first and second ultrasound scans. The process 1900 may then proceed to act 1908.

In act 1908, the processing circuitry may calculate, based on the second ultrasound data, volumetric blood flow through the blood vessel. In some embodiments, the processing circuitry may calculate spatial mean velocity from the second ultrasound data and multiply cross-sectional area of the blood vessel by spatial mean velocity to calculate the volumetric blood flow. In some embodiments, the processing circuitry may use deep learning to calculate spatial mean velocity from the second ultrasound data. The process 1900 may then proceed to act 1910.

In act 1910, the processing circuitry may calculate, based on the cross-sectional area of the blood vessel and the volumetric blood flow, the pulse wave velocity in the blood vessel. PWV may be calculated from the volumetric flow rate and cross-sectional area measurements by measuring the slope of a linear portion of a volumetric flow rate vs. cross-sectional area plot. In some embodiments, instead of/in addition to calculating PWV based on volumetric flow rate and cross-sectional area measured at an arterial site, the processing circuitry may calculate PWV using pulse wave imaging. Further description of measuring PWV has already been presented herein in the section entitled “Example Data Collection and Processing.”

Various inventive concepts may be embodied as one or more processes, of which examples have been provided. The acts performed as part of each process may be ordered in any suitable way. Thus, embodiments may be constructed in which acts are performed in an order different than illustrated, which may include performing some acts simultaneously, even though shown as sequential acts in illustrative embodiments. Further, one or more of the processes may be combined and/or omitted.

The terms “program,” “application,” or “software” are used herein in a generic sense to refer to any type of computer code or set of processor-executable instructions that may be employed to program a computer or other processor to implement various aspects of embodiments as discussed above. Additionally, according to one aspect, one or more computer programs that when executed perform methods of the disclosure provided herein need not reside on a single computer or processor, but may be distributed in a modular fashion among different computers or processors to implement various aspects of the disclosure provided herein.

Processor-executable instructions may be in many forms, such as program modules, executed by one or more computers or other devices. Generally, program modules include routines, programs, objects, components, data structures, etc., that perform particular tasks or implement particular abstract data types. Typically, the functionality of the program modules may be combined or distributed.

Also, data structures may be stored in one or more non-transitory computer-readable storage media in any suitable form. For simplicity of illustration, data structures may be shown to have fields that are related through location in the data structure. Such relationships may likewise be achieved by assigning storage for the fields with locations in a non-transitory computer-readable medium that convey relationship between the fields. However, any suitable mechanism may be used to establish relationships among information in fields of a data structure, including through the use of pointers, tags or other mechanisms that establish relationships among data elements.

Various aspects of the present disclosure may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Further, some actions are described as taken by a “operator” or “subject.” It should be appreciated that a “operator” or “subject” need not be a single individual, and that in some embodiments, actions attributable to an “operator” or “subject” may be performed by a team of individuals and/or an individual in combination with computer-assisted tools or other mechanisms. Further, it should be appreciated that, in some instances, a “subject” may be the same person as the “operator.” For example, an individual may be imaging themselves with an ultrasound device and, thereby, act as both the “subject” being imaged and the “operator” of the ultrasound device.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The terms “approximately” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having described above several aspects of at least one embodiment, it is to be appreciated various alterations, modifications, and improvements will readily occur to those skilled in the art. Such alterations, modifications, and improvements are intended to be object of this disclosure. Accordingly, the foregoing description and drawings are by way of example only.

What is claimed is:

1. An apparatus comprising an ultrasound-on-a-chip device configured to be bound to a user's wrist.

2. The apparatus of claim 1, wherein the ultrasound-on-a-chip device is waterproof.

3. The apparatus of claim 1, wherein the ultrasound-on-a-chip device is configured to perform both transverse and longitudinal ultrasound scanning of a blood vessel in the user's wrist without being rotated relative to the user's wrist.

4. The apparatus of claim 1, wherein the ultrasound-on-a-chip device comprises a two-dimensional array of capacitive micromachined ultrasonic transducers (CMUTs).

5. The apparatus of claim 1, wherein the ultrasound-on-a-chip device comprises a plurality of capacitive micromachined ultrasonic transducers (CMUTs) configured to emit ultrasound waves having a frequency between approximately 5-20 MHz.

6. The apparatus of claim 1, further comprising:
at least one wristband;
an ultrasound module containing the ultrasound-on-a-chip device and coupled to the at least one wristband; and
a coupling strip coupled to the ultrasound module and configured to couple the ultrasound module to the user's wrist.

7. The apparatus of claim 6, further comprising a primary module coupled to the at least one wristband.

8. The apparatus of claim 7, wherein the primary module comprises a display screen configured to display at least one of ultrasound data collected by the ultrasound-on-a-chip device, an ultrasound image generated from the ultrasound data, and data generated from the ultrasound data.

9. The apparatus of claim 1, further comprising:
at least one wristband configured to couple to a wristband of a wrist device;
an ultrasound module containing the ultrasound-on-a-chip device and coupled to the at least one wristband; and
a coupling strip coupled to the ultrasound module and configured to couple the ultrasound module to the user's wrist.

10. The apparatus of claim 9, further comprising a connection cable extending externally from the at least one wristband and configured to electrically connect the ultrasound module to the primary module.

11. The apparatus of claim 1, further comprising:
at least one wristband;
a primary module containing the ultrasound-on-a-chip device and coupled to the at least one wristband; and
a coupling strip coupled to the primary module and configured to couple the primary module to the user's wrist.

12. The apparatus of claim 11, further comprising a reservoir containing liquid or gel and configured to refresh the coupling strip.

13. The apparatus of claim 12, wherein the reservoir comprises a valve opening from the reservoir into the coupling strip and configured to enable flow of the liquid or gel from the reservoir to the coupling strip.

14. The apparatus of claim 13, wherein the valve is configured to enable flow of the liquid or gel from the reservoir to the coupling strip in response to mechanical pressure applied to a portion of the apparatus.

15. The apparatus of claim 13, further comprising:
processing circuitry configured to automatically trigger the valve to enable flow of the liquid or gel from the reservoir to the coupling strip.

16. The apparatus of claim 12, wherein the reservoir further comprises an input port configured to enable refilling of the reservoir with the liquid or gel.

17. The apparatus of claim 1, further comprising processing circuitry configured to generate a notification to reposition the ultrasound-on-a-chip device.

18. The apparatus of claim 17, further comprising processing circuitry configured to generate a notification to replace the coupling strip or refresh the coupling strip with liquid or gel.

19. The apparatus of claim 1, wherein the ultrasound-on-a-chip device is configured to transmit ultrasound data collected by the ultrasound-on-a-chip device to processing circuitry that is configured to analyze the ultrasound data using deep learning models.

20. The apparatus of claim 19, wherein the processing circuitry is configured to retrieve, from a server, ultrasound data collected by other ultrasound-on-a-chip devices and to use the ultrasound data collected by the other ultrasound-on-a-chip devices when training the deep learning models.

21. The apparatus of claim 1, wherein the ultrasound-on-a-chip device is configured to transmit ultrasound data of a blood vessel to processing circuitry that is configured to calculate pulse wave velocity in the blood vessel based on the ultrasound data of the blood vessel.

22. The apparatus of claim 1, wherein the apparatus further comprises:
a button; and
processing circuitry configured to trigger collection of ultrasound data by the ultrasound-on-a-chip device upon activation of the button.

23. An apparatus, comprising:
a wristband; and
an ultrasound-on-a-chip device coupled to the wristband.

24. The apparatus of claim 23, wherein the wristband comprises an interior surface and an exterior surface, and wherein the ultrasound-on-a-chip device is positioned on the interior surface of the wristband.

25.-39. (canceled)
